(19) United States
(12) Patent Application Publication
(10) Pub. No.: US 2005/0197343 A1
(43) Pub. Date: Sep. 8, 2005

(54) Utilization of heteroarene carboxamide as dopamine-d3 ligands for the treatment of cns diseases

(76) Inventors: Peter Gmeiner, Erlangen-Buckenhof (DE); Harald Hubner, Heroldsbach (DE); Karin Schlotter, Oettingen (DE)

Correspondence Address: NATH & ASSOCIATES, 1030 15th STREET, NW, 6TH FLOOR, WASHINGTON, DC, 20005, US

(21) Appl. No.: 10/519,487

(22) PCT Filed: Jul. 2, 2003
(86) PCT No.: PCT/EP03/07060

§ 371 Date: Apr. 27, 2005

(30) Foreign Application Priority Data
Jul. 4, 2002 (DE) 102 30 062.3
Jul. 10, 2002 (DE) 102 32 020.9

(51) Int. Cl. A61K 31/496; C07D 49/02; C07D 45/02

(52) U.S. Cl. 514/252.13; 514/254.11; 544/375

(57) ABSTRACT

The invention relates to neuroreceptor active N-[(4-phenyl-1-piperazinyl)alkyl]-substituted heteroarene carboxamide of general formula (I) and to structure analogous 2-ferrocenyl compounds of general formula (II) and the utilization thereof for the treatment of CNS diseases, for example, schizophrenia, different forms of depression, neurodegenerative disorders, sexual dysfunctions, cocaine, alcohol, opiate and nicotine addiction, in addition to glaucoma, cognitive disorders, restless leg syndrome, hyperactivity syndrome (ADHS), hyperprolactinemia, hyperprolactinoma, locomotion disorders associated with Parkinson's disease, treatment of L-DOPA and neuroleptic-induced locomotion disorders, for example, akathisia, rigor, dystonia and dyskinesia, wherein the substituents are defined in the description.

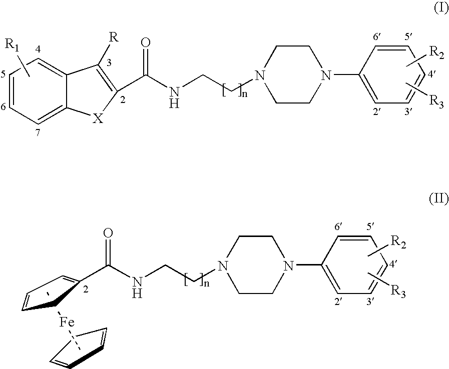

Dopamine is considered an important neurotransmitter of the central nervous system. Dopamine acts by binding to five different dopamine receptors. Owing to their morphology and the manner of signal transmission, these may be classified as D1-like (D1 and D5) and D2-like (D2, D3 and D4) receptors (Neve, K. A. The Dopamine Receptors. Humana Press, 1997). Especially the subtypes of the D2 family play an important role in regulatory processes of the central nervous system. While the D2 receptors are primarily expressed in the basal ganglia where they control neuromotoric circuits, D3 receptors are mainly found in the limbic system where emotional and cognitive processes are controlled. Disorders in the signal transduction of these receptors result in numerous neuropathological situations. Especially the D3 receptor is considered a promising target for the development of active ingredients to treat psychiatric diseases such as schizophrenia or unipolar depression, disturbances of consciousness as well as for the treatment of neurodegenerative diseases such as Parkinson's disease, but also for the treatment of drug addiction (Pulvirenti, L. et al. Trends Pharmacol. Sci. 2002, 23, 151-153).

Compounds having an aryl piperazine structure have already been described as dopamine receptor-active ligands (Robarge, M. J. J. Med. Chem. 2001, 44, 3175-3186). Benzamides and naphthamides having an aryl piperazine partial structure are also known as ligands of dopamine receptors (Perrone, R. J. Med. Chem. 1998, 41, 4903-4909; EP 0 779 284 A1). A phenyl piperazinyl naphthamide has recently been described as a selective D3-partial agonist showing promising activity in animal models which might be used for the treatment of cocaine addiction (Pilla, M. et al. Nature 1999, 400, 371-375).

For a few examples, aryl piperazinyl amides having oxygen-, sulfur- or nitrogen-containing heteroarenic acid components have been described (ES 2 027 898; EP 0 343 961; U.S. Pat No. 3,646,047; U.S. Pat. No. 3,734,915). Cyano-substituted and tellurium-containing derivatives with a ferrocenyl partial structure, on the other hand, are not known in literature.

In the course of our experiments concerning the effects of the structure of dopamine ligands we have discovered new compounds of the formulae (I) to (IV) which show hitherto unknown highly affine and highly selective binding characteristics to the D3 receptor in in vitro assays. These compounds could thus be valuable therapeutic agents for the treatment of diseases of the central nervous system such as schizophrenia, various forms of depression, neurodegenerative disorders, sexual dysfunction as well as cocaine, opiate and nicotine addiction.

Other specific areas of application are glaucoma, cognitive disorders, restless leg syndrome, hyperactivity syndrome (ADHS), hyperprolactinaemia, hypeprolactinoma, locomotion disorders associated with Parkinson's disease, treatment of L-DOPA- and neuroleptic-induced locomotion disorders, for example akathisia, rigor, dystonia and dyskinesias.

The subject matter of the present invention are derivatives of 2-heteroarene carboxylic acid amides having an aryl piperazinyl partial structure in the form of the free base and salts thereof as represented by the following formulae (I) and (II): 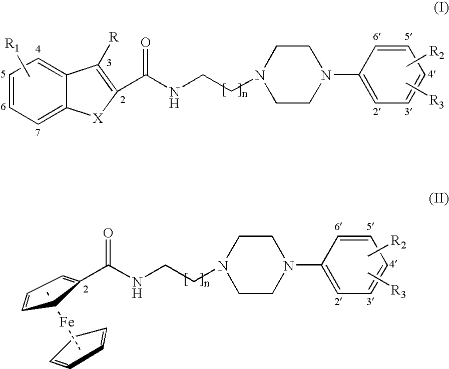
wherein in formula (I):
−n=1-4and
−R=hydrogen, alkyl or halogen and
(a) X=S or O:
   (i) when R₁ is hydroxy, alkyloxy, alkenyl, alkinyl, aryl, acyl, alkoxycarbonyl or cyano, each of R₂ and R₃ are independently selected from hydrogen, hydroxy, alkyloxy, alkyl, alkenyl, alkinyl, aryl, halogen, trifluoromethyl, acyl, alkoxycarbonyl and cyano,
   (ii) when R₁ is hydrogen, alkyl, halogen or trifluoromethyl, R₂ is selected from hydroxy, alkenyl, alkinyl, aryl, acyl, alkoxycarbonyl and cyano and R₃ is selected from hydrogen, hydroxy, alkyloxy, alkyl, alkenyl, alkinyl, aryl, halogen, trifluoromethyl, acyl, alkoxycarbonyl and cyano,
      or
(b) X=NH:
   R₁ is selected from hydrogen, hydroxy, alkyl, alkyloxy, alkenyl, alkinyl, aryl, trifluoromethyl, acyl, alkoxycarbonyl, halogen and cyano and each of R₂ and R₃ are selected independently from hydrogen, hydroxy, alkyloxy, alkyl, alkenyl, alkinyl, aryl, halogen, trifluoromethyl, acyl, alkoxycarbonyl and cyano, with the proviso that the compound is not N-4-(4-(2-methoxyphenyl)piperazine -1-yl)butyl-2-indolylcarbamide,
      or
(c) X=Te:
   R₁ is selected from hydrogen, hydroxy, alkyl, alkyloxy, alkenyl, alkinyl, aryl, halogen, trifluoromethyl, acyl, alkoxycarbonyl and cyano and each of R₂ and R₃ are selected independently from hydrogen, hydroxy, alkyloxy, alkyl, alkenyl, alkinyl, aryl, halogen, trifluoromethyl, acyl, alkoxycarbonyl and cyano.

In one embodiment of the invention, the following applies in formula (I):
n=1-4
   and
X=Te, when R=hydrogen, alkyl or halogen and R₁ is substituted by the radicals hydrogen, hydroxy, alkyl, alkyloxy, alkenyl, alkinyl, aryl, halogen, trifluoromethyl, acyl, alkoxycarbonyl or cyano and R₂ and R₃ are substituted individually or jointly by the radicals hydrogen, hydroxy, alkyloxy, alkyl, alkenyl, alkinyl, aryl, halogen, trifluoromethyl, acyl, alkoxycarbonyl or cyano, or
X=S or O, when R=hydrogen, alkyl or halogen and R₁ is substituted by the radicals hydroxy, alkyloxy, alkenyl, alkinyl, aryl, acyl, alkoxycarbonyl or cyano and R₂ and R₃ are substituted individually or jointly by the radicals hydrogen, hydroxy, alkyloxy, alkyl, alkenyl, alkinyl, aryl, halogen, trifluoromethyl, acyl, alkoxycarbonyl or cyano, or
X=S or O, when R=hydrogen, alkyl or halogen and R₁ is substituted by the radicals hydrogen, alkyl, halogen or trifluoromethyl and R₂ and R₃ are substituted individually or jointly by the radicals hydroxy, alkenyl, alkinyl, aryl, acyl, alkoxycarbonyl or cyano, or
X=NH, when R=hydrogen, alkyl or halogen and R₁ is substituted by the radicals hydroxy, alkyl, alkyloxy, alkenyl, alkinyl, aryl, trifluoromethyl, acyl, alkoxycarbonyl or cyano, it being required that alkyl and alkyloxy contain at least two carbon atoms, and R₂ and R₃ are substituted individually or jointly by the radicals hydrogen, hydroxy, alkyloxy, alkyl, alkenyl, alkinyl, aryl, halogen, trifluoromethyl, acyl, alkoxycarbonyl or cyano and alkyloxy comprises at least two carbon atoms.

In formula (II),

n=1-4 and R₁ and R₂ individually or jointly represent the radicals hydrogen, hydroxy, alkyloxy, alkyl, alkenyl, alkinyl, aryl, halogen, trifluoromethyl, acyl, alkoxycarbonyl or cyano.

In particular, the invention relates to physiologically acceptable salts of the compounds of the invention.

A skilled practitioner will be aware that, depending on the choice of substituents, optically active compounds may result. In that case, both the racemates and each of the pure enantiomeric forms are subject matters of the present invention.

The substituents listed in the description and the accompanying claims especially comprise the groups discussed below.

“Alkyl” may be a branched or unbranched alkyl group which preferably contains 1 to 10 carbon atoms, especially preferably 1 to 6 carbon atoms and most preferably 1, 2 or 3 carbon atoms, e.g. methyl, ethyl, n-propyl, iso-propyl, n-butyl, iso-butyl, s-butyl, t-butyl, n-pentyl, iso-pentyl, neopentyl, t-pentyl, 1-methylbutyl, 2-methylbutyl, 1-ethylpropyl, 1,2-dimethylpropyl and n-hexyl. Alkyl groups may additionally be substituted with one or more substituents, for example with halogen or one or more phenyl groups.

“Alkenyl” and “alkinyl” have at least one double or triple bond. They may be branched or unbranched and preferably comprise 2 to 6 carbon atoms. Alkenyls or alkinyls are preferably bound to the heteroarene or phenyl ring of the skeletal structure of the compound in such a manner that the double or triple bond is conjugated to the aromatic ring. Alkenyl and alkinyl may additionally be substituted with one or more substituents, preferably with phenyl; in that case the phenyl group is preferably located on the carbon atom 2 (if alkenyl or alkinyl is bound to the heteroarene or phenyl ring of the skeletal structure via the carbon atom 1). The alkenyls or alkinyls are preferably unsubstituted.

“Alkyloxy” is the —O-alkyl group, wherein alkyl is preferably selected from the groups listed above for “alkyl”. Preferably, alkyloxy is a C₁-C₆-alkyloxy group, especially methoxy. In another embodiment, alkyloxy may also be a C₂-C₆-alkyloxy group.

“Aryl” preferably is phenyl. Optionally, phenyl may also be substituted independently in one or more of the positions 2, 3 and 4, for example with alkoxy, trifluoromethyl or halogen, preferably with methoxy.

“Acyl” especially comprises the groups —C(O)-alkyl and —C(O)-aryl, wherein alkyl and aryl are preferably selected from the groups given for “alkyl” and “aryl” above, especially —C(O)-C₁-C₆-alkyl. For example, acyl is may be acetyl, propionyl, butyryl or —C(O)-phenyl.

“Alkoxycarbonyl” is the —C(O)—O-alkyl group, wherein alkyl is preferably selected from the groups listed for “alkyl” above. Preferably, alkoxycarbonyl is a (C₁-C₆-alkyl)oxy carbonyl group

“Halogen” is preferably fluorine, chlorine, bromine or iodine.

“Physiologically acceptable salts” comprise non-toxic addition salts of a base, especially of a compound of the formula (I) in the form of the free base with organic or inorganic acids. Examples for inorganic acids include HCl, HBr, sulfuric acid and phosphoric acid. Organic acids include acetic acid, propionic acid, pyruvic acid, butyric acid, α-, β- or γ-hydroxybutyric acid, valeric acid, hydroxyvaleric acid, capronic acid, hydroxycapronic acid, caprylic acid, capric acid, lauric acid, myristic acid, palmitic acid, stearic acid, glycolic acid, lactic acid, D-glucuronic acid, L-glucuronic acid, D-galacturonic acid, glycine, benzoic acid, hydroxybenzoic acid, gallic acid, salicylic acid, vanillic acid, cumaric acid, caffeic acid, hippuric acid, orotic acid, L-tartaric acid, D-tartaric acid, D,L-tartaric acid, meso-tartaric acid, fumaric acid, L-malic acid, D-malic acid, D,L-malic acid, oxalic acid, malonic acid, succinic acid, maleic acid, oxalacetic acid, glutaric acid, hydroxyglutaric acid, ketoglutaric acid, adipic acid, ketoadipic acid, pimelic acid, glutamic acid, aspartic acid, phthalic acid, propanetricarboxylic acid, citric acid, isocitric acid, methanesulfonic acid, toluenesulfonic acid and trifluoromethanesulfonic acid.

Compounds of the formula (I) wherein X is represented by NH, S or O may be named as preferred structures.

Preferred embodiments of the compounds of the formula (I) according to the invention are the following compounds of the general formulae (Ia) or (Ib): 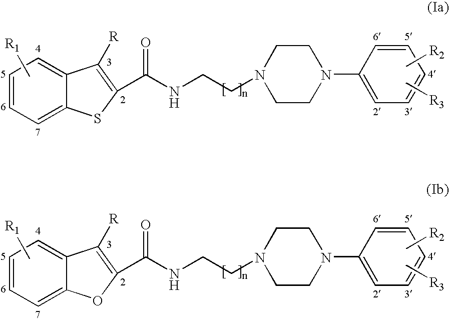
wherein:
n=1-4,
R=hydrogen, C₁-C₆-alkyl or halogen,
when R₁ is hydroxy, C₁-C₆-alkyloxy, C₂-C₆-alkenyl, C₂-C₆-alkinyl, phenyl that may optionally be substituted with a methoxy group or halogen, C₁-C₆-acyl, C₁-C₆-alkoxy carbonyl or cyano, each of R₂ and R₃ are independently selected from hydrogen, hydroxy, C₁-C₆-alkyloxy, C₁-C₆-alkyl, C₂-C₆-alkenyl, C₂-C₆-alkinyl, phenyl that may optionally be substituted with a methoxy group or halogen, halogen, trifluoromethyl, C₁-C₆-acyl, C₁-C₆-alkoxycarbonyl and cyano,
when R₁ is hydrogen, C₁-C₆-alkyl, halogen or trifluoromethyl, R₂ is selected from hydroxy, C₂-C₆-alkenyl, C₂-C₆-alkinyl, phenyl that may optionally be substituted with a methoxy group or halogen, C₁-C₆-acyl, C₁-C₆-alkoxycarbonyl and cyano, and R₃ is selected from hydrogen, hydroxy, C₁-C₆-alkyl, C₁-C₆-alkyloxy, C₂-C₆-alkenyl, C₂-C₆-alkinyl, phenyl that may optionally be substituted with a methoxy group or halogen, halogen, trifluoromethyl, C₁-C₆-acyl, C₁-C₆ alkoxycarbonyl and cyano,
   and pharmaceutically acceptable salts thereof, fluorine, chlorine and bromine being preferred halogen substituents.

Another preferred embodiment of the compounds of the formula (I) according to the invention are the following compounds of the general formula (Ic): 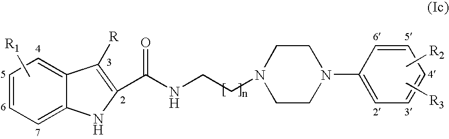
wherein:
n=1-4,
R=hydrogen, C₁-C₆-alkyl or halogen,
R₁ is selected from hydrogen, hydroxy, C₁-C₆-alkyl, C₁-C₆-alkoxy, C₂-C₆-alkenyl, C₂-C₆-alkinyl, phenyl that may optionally be substituted with a methoxy group or halogen, trifluoromethyl, C₁-C₆-acyl, C₁-C₆-alkoxycarbonyl, fluorine, chlorine, bromine and cyano,
each of R₂ and R₃ are independently selected from hydrogen, hydroxy, C₁-C₆-alkyl, C₁-C₆-alkyloxy, C₂-C₆-alkenyl, C₂-C₆-alkinyl, phenyl that may optionally be substituted with a methoxy group or halogen, halogen, trifluoromethyl, C₁-C₆-acyl, C₁-C₆-alkoxycarbonyl and cyano,
   and pharmaceutically acceptable salts of these compounds, with the proviso that the compound is not N-4-(4-(2-methoxyphenyl)piperazine-1-yl)butyl-2-indolylcarbamide.

In another preferred embodiment of the invention, the following applies for compounds of the general formula (Ic):

(a) when R₁ is hydroxy, C₂-C₆-alkenyl, C₂-C₆-alkinyl, phenyl that may optionally be substituted with a methoxy group or halogen, trifluoromethyl, C₁-C₆-acyl, C₁-C₆-alkoxycarbonyl or cyano, each of R₂ and R₃ are independently selected from hydrogen, hydroxy, C₁-C₆-alkyl, C₁-C₆-alkyloxy, C₂-C₆-alkenyl, C₂-C₆-alkinyl, phenyl that may optionally be substituted with a methoxy group or halogen, halogen, trifluoromethyl, C₁-C₆-acyl, C₁-C₆-alkoxycarbonyl and cyano, and

(b) when R₁ is hydrogen, C₁-C₆-alkyl, C₁-C₆-alkyloxy or halogen, R₂ is selected from hydroxy, C₂-C₆-alkenyl, C₂-C₆-alkinyl, phenyl that may optionally be substituted with a methoxy group or halogen, C₁-C₆-acyl, C₁-C₆-alkoxycarbonyl and cyano, and R₃ is selected from hydrogen, hydroxy, C₁-C₆-alkyl, C₁-C₆-alkyloxy, C₂-C₆-alkenyl, C₂-C₆-alkinyl, phenyl that may optionally be substituted with a methoxy group or halogen, halogen, trifluoromethyl, C₁-C₆-acyl, C₁-C₆-alkoxycarbonyl and cyano.

In another preferred embodiment of the invention, the following applies for the compounds of the formulae (I), (Ia), (Ib), and (Ic):
the substituent R₁ is in position 5 or 6 of the heterocycle, and
the substituents R₂ and R₃ are in the positions 2 or 3, respectively, or in the positions 2 or 4, respectively, of the phenyl ring; the respective other substituent being in position 2 of the phenyl ring in the event that one of the two substituents R₂ and R₃ is a hydrogen atom.

In a particularly preferred embodiment of the invention, n=3 in the compounds of the formulae (I), (Ia), (Ib), and (Ic).

Preferred compounds of the general formula (II) as defined above are those wherein each of R₁ and R₂ is independently selected from hydrogen, hydroxy, C₁-C₆-alkyloxy, C₁-C₆-alkyl, C₂-C₆-alkenyl, C₂-C₆-alkinyl, aryl, fluorine, chlorine, bromine, trifluoromethyl, C₁-C₆-acyl, C₁-C₆-alkoxycarbonyl and cyano.

Specific compounds of the general formula (II) are
(B16): N-4-(4-(2-methoxyphenyl)piperazine-1-yl)butyl-2-ferrocenylcarbamide and
(B17): N-4-(4-(2,3-dichlorophenyl)piperazine-1-yl)butyl-2-ferrocenylcarbamide

Another aspect of the present invention relates to compounds of the general formula (IV): 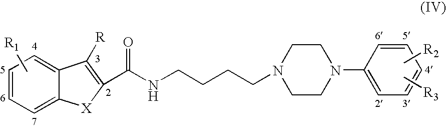
wherein:
X=S, NH or O,
R is selected from hydrogen, C₁-C₆-alkyl, fluorine, chlorine and bromine,
R₁ is selected from hydrogen, C₁-C₆-alkoxy, C₁-C₆-alkyl, fluorine, chlorine, bromine, trifluoromethyl and cyano, R₁ being in position 5 or 6 of the heterocycle,
R₂ and R₃ are independently selected from hydrogen, C₁-C₆-alkyloxy, C₁-C₆-alkyl, fluorine, chlorine, bromine and trifluoromethyl, R₂ and R₃ being in the positions 2 or 3, respectively, or in the positions 2 or 4, respectively, of the phenyl ring, and the respective other substituent being in position 2 of the phenyl ring in the event that one of the two substituents R₂ and R₃ is a hydrogen atom
   and pharmaceutically acceptable salts of this compound with the proviso that the compound is not N-4-(4-(2-methoxyphenyl)piperazine-1-yl)butyl-2-indolylcarbamide.

A preferred aspect of the invention are compounds of the general formula (IV) as defined above, wherein
when X=NH, then R₁ is selected from hydrogen, C₁-C₃-alkyloxy, C₁-C₃-alkyl, fluorine, chlorine, bromine and cyano, and
when X=S or O then R₁ is selected from hydrogen, C₁-C₃-alkyl, fluorine, chlorine, bromine, cyano and trifluoromethyl.

The following compounds are specific embodiments of the compounds according to the invention:
(B18): N-4-(4-(2-methoxyphenyl)piperazine-1-yl)butyl-5-cyano-2-benzo[b]thiophenylcarbamide
(B19): N-4-(4-(2,3-dichlorophenyl)piperazine-1-yl)butyl-5-cyano-2-benzo[b]thiophenylcarbamide
(B20): N-4-(4-(2-methoxyphenyl)piperazine-1-yl)butyl-6-cyano-2-benzo[b]thiophenylcarbamide
(B21): N-4-(4-(2 ,3 -dichlorophenyl)piperazine-1-yl)butyl-6-cyano-2-benzo[b]thiophenylcarbamide
(B1): N-4-(4-(2-methoxyphenyl)piperazine-1-yl)butyl-2-benzo[b]-thiophenylcarbamide
(B2): N-4-(4-(2,3-dichlorophenyl)piperazine-1-yl)butyl-2-benzo[b]-thiophenylcarbamide
(B22): N-4-(4-(2-methoxyphenyl)piperazine-1-yl)butyl-5-bromo-2-benzo[b]thiophenylcarbamide
(B23): N-4-(4-(2,3-dichlorophenyl)piperazine-1-yl)butyl-5-bromo-2-benzo[b]thiophenylcarbamide
(B10): N-4-(4-(2,3-dichlorophenyl)piperazine-1-yl)butyl-2-indolylcarbamide
(B11): N-4-(4-(2-methoxyphenyl)piperazine-1-yl)butyl-5-cyano-2-indolylcarbamide
(B 12): N-4-(4-(2-methoxyphenyl)piperazine-1-yl)butyl-5-bromo-2-indolylcarbamide
(B 13): N-4-(4-(2-methoxyphenyl)piperazine-1-yl)butyl-6-cyano-2-indolylcarbamide
(B 14): N-4-(4-(2,3-dichlorophenyl)piperazine-1-yl)butyl-5-bromo-2-indolylcarbamide
(B 15): N-4-(4-(2,3-dichlorophenyl)piperazine-1-yl)butyl-6-cyano-2-indolylcarbamide
(B25): N-4-(4-(2,3-dichlorophenyl)piperazine-1-yl)butyl-5-cyano-2-indolylcarbamide
(B7): N-4-(4-(2-methoxyphenyl)piperazine-1-yl)butyl-5-cyano-2-benzo[b]furanylcarbamide
(B3): N-4-(4-(2-methoxyphenyl)piperazine-1-yl)butyl-2-benzo[b]-furanylcarbamide
(B4): N-4-(4-(2,3-dichlorophenyl)piperazine-1-yl)butyl-2-benzo[b]furanylcarbamide
(B5): N-4-(4-(2-methoxyphenyl)piperazine-1-yl)butyl-5-bromo-benzo[b]furanylcarbamide
(B6): N-4-(4-(2,3-dichlorophenyl)piperazine-1-yl)butyl-5-bromo-2-benzo [b]furanyl-carbamide
(B8): N-4-(4-(2-methoxyphenyl)piperazine-1-yl)butyl-2-benzo[b]tellurophenylcarbamide
(B9): N-4-(4-(2,3-dichlorophenyl)piperazine-1-yl)butyl-2-benzo[b]tellurophenylcarbamide
   and pharmaceutically acceptable salts of these compounds.

Especially the compounds of the formulae (I), (Ia), (Ib), (Ic), (II) and (IV) as defined above are suitable for therapeutic use as dopamine D3 ligands. Particular preference is given to compounds of the general formula (I) or pharmaceutically acceptable salts thereof, wherein X=NH, S or O, and to compounds of the formulae (Ia), (Ib), (Ic) and (IV) or pharmaceutically acceptable salts thereof.

The term “D3 ligands with high affinity” comprises compounds which show binding to human dopamine D3 receptors having a Ki value of preferably not more than 10 nM, especially preferably not more than 1 nM, in a radio ligand experiment (cf. Hübner, H. et al. J. Med. Chem. 2000, 43, 756-762, and the following section “Biological Activity”).

One aspect of the present invention relates to selective D3 ligands. The term “selective D3 ligands” comprises compounds with a Ki value in the radio ligand experiment for the D3 receptor as described in the following section “Biological Activity” which is lower by a factor of at least 10 for at least five of the seven following receptors: dopamine receptors D1, D2long, D2short and D4.4, serotonin receptors 5-HT1A and 5-HT2 and alpha-1-adrenoreceptor. Another aspect of the invention relates to dopamine D3 ligands with high selectivity. The term “D3 ligands with high selectivity” comprises compounds with a Ki value in the radio ligand experiment for the D3 receptor as described in the following section “Biological Activity” which is lower by a factor of at least 100 for at least three and preferably all of the of the dopamine receptors D1, D21long, D2short and D4.4.

D3 ligands may show agonistic, antagonistic or partial-agonistic activity on the D3 receptor. The respective intrinsic activities of the compounds of the invention may be measured in mitogenesis assays as described in literature (Hübner, H. et al. J. Med. Chem. 2000, 43, 4563-4569, and Löber, S. Bioorg. Med. Chem. Lett. 2002, 12.17, 2377-2380). Depending on the pathophysiology of the underlying disease, a stronger agonistic, stronger antagonistic or partial-agonistic activity may be desirable. The present invention therefore permits an excellent fine-tuning of the desired activity.

Therefore, a therapeutic agent comprising one or more of the compounds of the general formulae (I), (Ia), (Ib), (Ic), (II) and (IV) or one of the specific compounds as defined above, optionally in the form of a pharmaceutically acceptable salt, is a further subject matter of the invention. Preferably, this includes one or more of the compounds of the general formulae (I), (Ia), (Ib), (Ic) and (IV) or pharmaceutically acceptable salts thereof, wherein X=NH, S or O.

The invention also relates to the use of one or more of the compounds of the general formulae (I), (Ia), (Ib), (Ic), (II) and (IV) or one of the specific compounds listed, optionally in the form of a pharmaceutically acceptable salt, for the treatment, including therapy and prevention, of the indications given here and for preparing a therapeutic agent for said indications.

It is preferred to choose those compounds of the invention which are selective D3 ligands for preparing therapeutic agents. D3 ligands with high selectivity are especially preferred.

The compounds of the invention have potential use in the therapy or prevention of a number of disorders, especially those accompanied by a dysfunction of the dopamine metabolism or the dopaminergic signal cascade.

Examples for such disorders are cocaine, alcohol, opiate and nicotine addiction; neurodegenerative disorders, especially Parkinson's disease; sexual dysfunction, especially male erectile dysfunction; depression, especially endogenous monophase depression (“major depression”) and schizophrenia.

Other examples amenable to therapy or prevention with the compounds of the invention are hyperprolactinaemia; hyperprolactinoma; glaucoma; cognitive disorders; restless leg syndrome; hyperactivity syndrome (ADHS); locomotion disorders associated with Parkinson's disease, e.g. rigor, dystonia and dyskinesia; L-DOPA-induced disorders, such as anxiety, disturbed sleep, psychoses, dyskinesias and dystonias; idiopathic dystonias, especially Segawa syndrome; neuroleptic-induced (tardive) dyskinesia, dystonia and akathisia.

The compounds of the invention are particularly well suited to prepare a therapeutic agent for treating DOPA-sensitive locomotion disorders. Such locomotion disorders may be dyskinesias, dystonias, rigor and tremor, for example. The term “DOPA-sensitive” is understood to mean that the locomotion disorder may be influenced favourably by the administration of drugs which influence the dopaminergic signal transmission. A typical example is the Segawa syndrome, an idiopathic dystonia where the response to L-DOPA may be used as a diagnostic criterion.

A preferred use is the preparation of a therapeutic agent for the treatment of dyskinesias and dystonias which may occur spontaneously in connection with Parkinson's disease, but may also be induced by drugs. Among drug-induced dyskinesias and dystonias, especially those induced by neuroleptics and dopamine antagonists or dopamine agonists or L-DOPA may be mentioned.

In addition, the therapeutic agents may be used in medication-assisted ablactation after a pregnancy.

Finally, the therapeutic agents of the invention may be used as a compound preparation for simultaneous or sequential administration, depending on the disease to be treated.

For example, one unit offered for sale which contains L-DOPA medication for the treatment of Parkinson's disease may also comprise a pharmaceutical preparation which contains one of the compounds of the invention, for example with a high-selectivity, partial-agonistic profile of action. L-DOPA and the compound of the invention may be present in the same pharmaceutical formulation, e.g. a compound tablet or in different units of application, e.g. in the form of two separate tablets. As needed, the two active ingredients may be administered simultaneously or at different times

In a compound preparation, sequential administration may be achieved by providing a form of administration, for example an oral tablet, having two different layers with different release profiles for the various pharmaceutically active components. A skilled practitioner will appreciate that various forms of administration and application schemes are conceivable within the context of the invention all of which are subject matters thereof.

One embodiment of the invention therefore relates to a therapeutic agent which contains L-DOPA or a neuroleptic as well as a compound of the invention for the simultaneous or sequential administration to a patient.

In general, the therapeutic agents of the invention consist of a pharmaceutical composition which, in addition to the D3 ligands as described above, contains at least one pharmaceutically acceptable carrier or excipient.

A skilled practitioner will appreciate that, depending on the intended route of application, the pharmaceutical formulation may take different forms. For example, the pharmaceutical formulation may be adapted for intravenous, intramuscular, intracutaneous, subcutaneous, oral, buccal, sublingual, nasal, transdermal, inhalational, rectal or intraperitoneal administration.

Suitable formulations and pharmaceutical carriers and excipients, respectively, amenable for this purpose such as fillers, disintegrants, binders, lubricants, stabilisers, flavouring agents, antioxidants, preservatives, dispersants or solvents, buffers or electrolytes are known to practitioners skilled in the field of pharmaceuticals and are described in standard works, for example those by Sucker, Fuchs and Speiser (“Pharmazeutische Technologie”, Deutscher Apotheker Verlag, 1991) and Remington (“The Science and Practice of Pharmacy”, Lippincott, Williams & Wilkins, 2000).

In a preferred embodiment of the invention, the pharmaceutical compositions containing the compounds according to the invention may be administered orally and may be present, for example, as a capsule, tablet, powder, granulate, coated tablet or in liquid form.

The formulation may be prepared as a rapid-release form of administration if fast onset of action is desired. Suitable oral formulations are described in EP 0 548 356 or EP 1 126 821.

If protracted release is desired, on the other hand, a formulation with delayed release of the active ingredient may be selected. Such oral formulations are also known from the prior art.

Alternative pharmaceutical preparations may, for example, be solutions for infusion or injection, oils, suppositories, aerosols, sprays, plasters, micro-capsules or micro-particles.

Another subject matter of the invention is the use of a compound of the general formula (III): 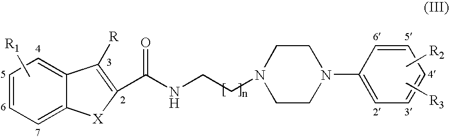
wherein:
n=1-4 and X=S, O or NH, when R=hydrogen, alkyl or halogen and R₁ are substituted by the radicals hydrogen, alkyl, halogen, trifluoromethyl and each of R₂ and R₃ is substituted individually or jointly by the radicals hydrogen, hydroxy, alkyloxy, alkyl, alkenyl, alkinyl, aryl, halogen, trifluoromethyl, acyl, alkoxycarbonyl or cyano, for preparing a pharmaceutical agent for the treatment of one of the following diseases or disorders:

cocaine, alcohol, opiate and nicotine addiction; schizophrenia; various forms of depression, especially endogenous monophase depression (“major depression”) or depressive phases of bipolar (manic-depressive) disorders; neurodegenerative disorders, especially Parkinson's disease; sexual dysfunction; hyperprolactinaemia; hyperprolactinoma; glaucoma; cognitive disorders; restless leg syndrome; hyperactivity syndrome (ADHS); locomotion disorders associated with Parkinson's disease, e.g. rigor, dystonia and dyskinesia; L-DOPA-induced disorders, such as anxiety, disturbed sleep, psychoses, dyskinesias and dystonias; idiopathic dystonias, especially Segawa syndrome; neuroleptic-induced (tardive) dyskinesia, dystonia and akathisia.

In particular, a compound of the general formula (III) may be used to prepare a therapeutic agent for the treatment DOPA-sensitive locomotion disorders. These may occur spontaneously in connection with Parkinson's disease, but may also be induced by drugs. Among drug-induced locomotion disorders, especially those induced by neuroleptics or dopamine antagonists or L-DOPA-induced dyskinesias and dystonias may be mentioned.

It is preferred to use compounds of the general formula (III) for preparing the therapeutic agents of the invention, wherein
R is selected from hydrogen, C₁-C₆-alkyl, fluorine, chlorine and bromine,
R₁ is selected from hydrogen, C₁-C₆-alkoxy, C₁-C₆-alkyl, fluorine, chlorine, bromine and trifluoromethyl, and
each of R₂ and R₃ is independently selected from hydrogen, C₁-C₆-alkyloxy, C₁-C₆-alkyl, fluorine, chlorine, bromine and trifluoromethyl.

It is also preferred to use compounds of the formula (III), wherein
the substituent R₁ is in position 5 or 6 of the heterocycle, and
the substituents R₂ and R₃ are in the positions 2 or 3, respectively, or in the positions 2 or 4, respectively, of the phenyl ring; the respective other substituent being in position 2 of the phenyl ring in the event that one of the two substituents R₂ and R₃ is a hydrogen atom.

A preferred compound of the general formula (III) for preparing the therapeutic agents of the invention, especially for the treatment of L-DOPA-induced dyskinesias, is the following compound:
(B24): N-4-(4-(2-methoxyphenyl)piperazine-1-yl)butyl-2-indolylcarbamide

The compounds of the formulae (I) bis (IV) were prepared according to methods described in literature (Glennon, R. A et al. J. Med. Chem. 1988, 31, 1968-1971).

For this purpose, acid derivatives of the type (A) which were either available commercially or were synthesised as prescribed in literature were activated in the form of their carboxylic acid chlorides and reacted with the free base of the type (B) to obtain derivatives of the formula (I) (including (Ia), (Ib) and (Ic), (III) or (IV): 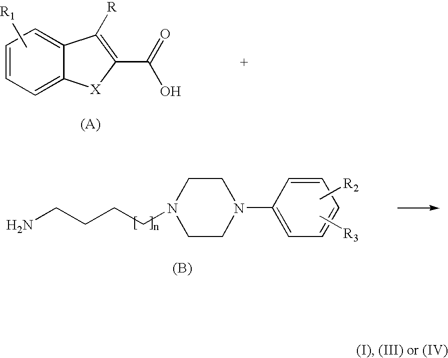
wherein n, R, R₁, R₂ and R₃ are as defined above for (I), (III) and (IV), respectively.

As an alternative to the above-mentioned method of activating the acid derivatives, it is possible to use other reactions, for example activation of acids by hydroxyazabenzotriazole (Kienhofer, A. Synlett 2001, 1811-1812). For example, the compounds of the formula (II) may be prepared by activating ferrocene-2-carboxylic acid with HATU followed by a reaction with the bases of the type (B).

Commercially available 2-methoxy- or 2,3-dichlorophenyl piperazines may be alkylated with bromobutyl phthalimide in xylene to prepare the aryl piperazinyl amines of the type (B). Subsequent hydrazinolysis of the phthalimide-substituted structures provides the primary amines of the type (B). This is illustrated by the following exemplary reaction scheme:

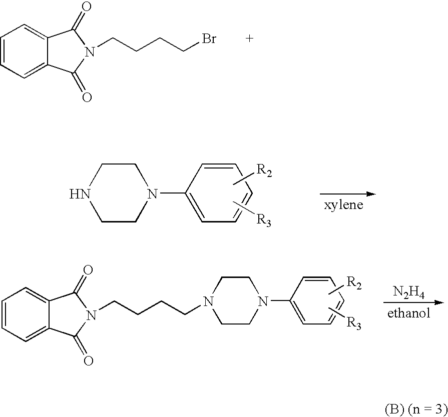

### EXAMPLES

### Synthesis of the Amines of the Type (B)

### 4-(4-(2, 3-dichlorophenyl)piperazine-1-yl)butylamine

2,3 g (10 mmol) of the free base of the 2,3-dichlorophenyl piperazine are dissolved in 10 ml of xylene and heated to 70° C. Then 1.4 g (5 mmol) of 4-bromobutyl phthalimide are dissolved in 20 ml of xylene and slowly dropped into the piperazine solution. The reaction mixture is heated at 125° C. for 24 hours. After cooling to 0° C., the mixture is filtered and the filtrate evaporated. The resulting N-4-(4-(2,3-dichlorophenyl)piperazine-1-yl)butylphthalimide is purified by flash chromatography on SiO₂ with ethyl acetate. Yield: 4.0 g (=92%)

A solution of 80% hydrazine hydrate (0.45 ml, 2.5 eq) in 5 ml of ethanol is dropped into a suspension of N-4-(4-(2,3-dichlorophenyl)piperazine- 1-yl)butylphthalimide in 40 ml of ethanol. The mixture is heated at reflux for 3 hours, then cooled to room temperature, the precipitated solid filtered off, and the ethanolic solution evaporated in vacuo.

Purification by flash chromatography with CH₂Cl₂-MeOH-Me₂EtN:90-8-2 provides the base 4-(4-(2,3-dichlorophenyl)piperazine-1-yl)butylamine in a yield of 900 mg (=60%).

MS: m/z 301(M+); IR: (NaCl): 3397; 2939; 2817; 1641; 1572; 1500; 1482; 1452; 1376; 1240; 1152; 1118; 1023; 917; 791; 749; 698; 661. 1H-NMR (CDCl₃, 360 MHz) δ (ppm): 1.48-1.64 (m, 4H,CH₂-CH₂); 2.41-2.46 (t, J=7.6, 2H, CH₂N); 2.64 (m, 4H, pip); 2.72-2.76 (m, 2H, CH₂NCO); 3.07 (m, 4H, pip); 6.93-6.99 (m, 1H, H₅, phenyl); 7.11-7.17 (m, 2H, H₄, H₆, phenyl).

### Example 1

### N-4-(4-(2-methoxyphenyl)piperazine-1-yl)butyl-2-benzo[b]thiophenylcarbamide

0,4 mmol of benzothiophene-2-carboxylic acid (0.071 g) are dissolved in 4 ml of dry toluene and 4 ml of dry chloroform. 0.02 ml of dry DMF and 0.11 ml (1.51 mmol) of SOCl₂ are added, followed by heating in an oil bath to 90° C. for 30 minutes. The solvent is then removed by rotation and dried in a fine vacuum. The acid chloride is dissolved in 40 ml of chloroform and added with stirring at 0° C. to a solution of 0.4 mmol of 4-(4-(2-methoxyphenyl)piperazine-1-yl)butylamine (0.105 g) and 0.17 ml of Et₃N in 5 ml of chloroform. After a reaction time of 14 hours, the reaction is washed with aqueous NaHCO₃ solution, the organic solvent dried with MgSO4 and evaporated in vacuo. Purification by flash chromatography on silica gel CH₂Cl₂-MeOH:9-1 provides 114 mg (68% yield in 2 reaction steps) of N-4-(4-(2-methoxyphenyl)piperazine -1-yl)butyl-2-benzo[b]thiophenylcarbamide.

Smp.: 147° C.; MS: m/z 423 (M⁺); IR (NaCl): 3316; 2938; 2817; 1735; 1629; 1544; 1500; 1241; 1026; 731. ¹H-NMR(CDC1₃, 360MHz) 6 (ppm):1.65-1.74 (m, 4H, CH₂-CH₂); 2.47 (t, 2H, CH₂N, J=6.7 Hz); 2.65 (m, 4H, pip); 3.08 (m, 4H, pip); 3.48-3.53 (m, 2H, CH₂NCO), 3.85 (s, 3H, OCH₃); 6.72 (t, 1H, NH, J=4.3 Hz); 6.84-7.01 (m, 4H, phenyl); 7.36-7.44 (m, 2H, H₅, H₆); 7.76 (s, 1H, H₃); 7.79-7.85 (m, 2H, H₇, H₄). ¹³C-NMR (CDCl₃, 90 MHz) δ (ppm): 162.4; 152.3; 141.2; 140.7; 139.1; 138.7; 126.2; 125.0; 124.9; 122.9; 122.7; 120.9; 118.2; 111.2; 57.9; 55.3; 53.4; 50.5; 40.0; 27.4; 24.3. C H N (%): C₂₄H₂₉N₃O₂S; Calc.: C 68.05; H 6.90; N 9.92; S 8.15; Found: C 68.11; H 6.95; N 9.93; S 8.09.

### Example 2

### N-4-(4-(2, 3-dichlorophenyl)piperazine-1-yl)butyl-2-benzo[b]thiophenylcarbamide

Synthesis Analogous to Example 1.

Yield: 126 mg (68% in 2 reaction steps)

Smp.: 153° C. MS: m/z 462 (M⁺). IR (NaCl): 3298; 2967; 2934; 2809; 1640; 1599; 1576; 1530; 1442; 1420; 1301; 1167; 1131; 962; 882; 808; 781; 712. ¹H-NMR: (CDCl₃, 360 MHz) δ (ppm): 1.63-1.76 (m, 4H, CH₂-CH₂); 2.48 (t, J=6.9 Hz, 2H, CH₂N); 2.66 (m, 4H, pip); 3.05 (m, 4H, pip); 3.49-3.54 (m, 2H, CH₂NCO); 6.79 (br, t, J=5.3 Hz, 1H, NH); 6.84-6.86 (dd, J =1.6 Hz, J =7.5 Hz, 1H, phenyl); 7.08-7.16 (m, 2H, phenyl); 7.37-7.44 (m, 2H, H₅, H₆); 7.77-7.78 (s, 1H, H₃); 7.80-7.90 (m; 2H, H₄, H₇). ¹³C-NMR (CDCl₃, 90 MHz) δ (ppm): 157.9; 156.1; 152.3; 151.1; 141.2; 129.9; 127.8; 123.0; 121.0; 118.2; 113.0; 112.3; 106.6; 109.7; 107.9; 57.9; 53.5; 50.5; 39.4; 27.4; 24.3. C H N (%): C₂₃H₂₅Cl₂N₃OS Calc.: C 60.25; H 6.11; N 8.78; Found: C 59.94; H 6.04; N 8.81.

### Example 3

### N-4-(4-(2-methoxyphenyl)piperazine-1-yl)butyl-2-benzo[b]furanylcarbarnide

Synthesis Analogous to Example 1.

Yield: 75.2 mg (56% yield % in 2 reaction steps)

Smp.: 121° C. MS: m/z431 (M⁺). IR(NaCl): 3311.2; 3060; 2937; 2815; 2216; 1654; 1592; 1500; 1321; 1240; 1178; 1145;748. ¹H-NMR (CDCl₃, 360 MHz) δ (ppm): 1.67-1.74 (m, 4H, CH₂-CH₂); 2.49 (t, 2H, CH₂N, J=6.9 Hz); 2.69 (m, 4H, pip); 3.13 (m, 4H, pip); 3.56-3.50 (m, 2H, CH₂NCO); 3.86 (s, 3H, OCH₃); 6.85-6.87 (m, 1H, phenyl); 6.90-6.93 (m, 2H phenyl); 6.99-7.02 (m, 2H, phenyl and NH); 7.26-7.31 (m, 1H, H₅); 7.37-7.42 (m, 1H, H₆); 7.46-7.48 (m, 2H, H₄, H₃); 7.77-7.79 (m, 1H, H₇). ¹³C-NMR (CDCl₃, 90 MHz) δ (ppm): 158.9; 154.7; 152.3; 148.9; 141.3; 127.7; 126.7; 123.6; 122.9; 122.7; 120.9; 118.2; 11 1.7; 11 1.2; 110.2; 58.0; 55.3; 53.5; 50.5; 39.2; 27.5; 24.3. CHN (%): C₂₄H₂₉N₃O₃.0.3 H₂0; Calc.: C 69.81; H 7.23; N 10.18; Found: C 69.84; H 7.33; N 10.21.

### Example 4

### N-4-(4-(2, 3-dichlorophenyl)piperazine-1-yl)butyl-2-benzo[b]furanylcarbamide

Synthesis Analogous to Example 1.

Yield: 105 mg (58% yield in 2 reaction steps) Smp.: 150° C. MS: m/z 446 (M+). IR (NaCl): 3310; 2939; 2819; 1652; 1595; 1577; 1520; 1448; 1421; 1299; 1257; 1241; 1176; 1141; 1044; 960; 908; 780; 748; 713, 669, 645. ¹H-NMR (CDCl₃, 360 MHz) δ (ppm): 1.67-1.75 (m, 4H, CH₂-CH₂); 2.52 (t, J=6.7 Hz, 2H, CH₂N); 2.69 (m, 4 H, pip); 3.13 (m, 4H, pip); 3.51-3.56 (m, 2H, CH₂NCO); 6.92-6.95 (dd, J=2.3 Hz, 7.3 Hz, 1H, phenyl); 7.00 (brt, J=4.3 Hz, 1H, NHCO); 7.10-7.17 (m, 2H, phenyl); 7.26-7.31 (m, 1H, H₄); 7.38-7.43 (m, 1H, H₆); 7.46-7.48 (m, 2H, H₃, H₅); 7.66-7.68 (m; 1H, H₇). ¹³C-NMR (CDCl₃, 90 MHz) δ (ppm): 158.9; 154.7; 151.2; 148.2; 134.0; 127.7; 127.5; 127.4; 126.8; 124.6; 123.7; 122.7; 118.6; 111.6; 110.2; 57.9; 53.3; 51.1; 39.2; 27.5; 24.2. CHN(%): C₂₃H₂₅CI₂N₃O₂ Calc.: C 61.89; H 5.65; N 9.41; Found: C 61.74; H 5.86; N 9.05.

### Example 5

### N-4-(4-(2-methoxyphenyl)piperazine-1 -yl)butyl-5-brom-2-benzo[b]furanylcarbamide

Synthesis analogous to example 1. 5-Bromo-2-benzo[b]furanylcarboxylic acid was prepared according to literature (Dann, O. Liebigs Ann. Chem. 1986, 438-455).

Yield: 107.8 mg (56% yield in 2 reaction steps)

Smp.: 124° C. MS m/z 485 (M⁺). IR (NaCl): 3450.0; 3289.9; 3068.2; 2927.4; 2765; 1650; 1567; 1535; 1500; 1438; 1238; 1178; 1022; 802; 748. ¹H-NMR (CDCl₃, 360 MHz) δ (ppm): 1.67-1.74 (m, 4H, CH₂-CH₂); 2.49 (t, J=6.9 Hz, 2H, CH₂N); 2.69 (m, 4H, pip); 3.13 (m, 4H, pip); 3.56-3.60 (m, 2H, CH₂NCO); 3.86 (s, 3H, OCH₃); 6.85-6.87 (m, 1H, phenyl); 6.91-6.93 (m, 2H, phenyl); 6.97-7.00 (m, 1H, phenyl); 7.00 (brt, J=4.8 Hz, 1H, NH); 7.32-7.35 (d, J=8.9 Hz, 1H, H₄); 7.38-7.39 (m, 1H, H₃); 7.48 (dd, J=8.7 Hz, J=2.0 Hz, 1H, H₆); 7.79-7.80 (m, 1H, H₇). ¹³C-NMR (CDCl₃, 90 MHz) δ (ppm): 158.4; 153.3; 152.3; 150.1; 141.2; 129.7; 129.6; 125.2; 122.9; 120.9; 120.9; 118.2; 116.7; 113.1; 111.2; 109.4; 64.8; 57.9; 55.3; 53.5; 50.4; 39.3; 27.5; 24.3. CHN (%): C₂₄H₂₉BrN₃O₂; Calc.: C 59.26; H 5.80; N 8.64; Found: C 59.05; H 5.81; N 8.68.

### Example 6

### N-4-(4-(2,3-dichlorophenyl)piperazine-1-yl)butyl-5-bromo-2-benzo[b]furanylcarbarnide

Synthesis Analogous to Example 5.

Yield: 102 mg (47% yield in 2 reaction steps)

Smp.: 145° C. MS m/z 524 (M⁺); IR (NaCl): 3400; 2937; 2815; 2227; 1666;: 1594; 1527; 1500; 1294; 1240; 1141; 1118; 1025; 842; 746. ¹H-NMR (CDCl₃, 360 MHz) δ (ppm): 1.63-1.74 (m, 4H, CH₂-CH₂); 2.49-2.52 (t, J=6,7 Hz, 2H, CH₂N); 2.68 (m, 4H, pip); 3.09 (m, 4H, pip); 3.49-3.56 (m, 2H, CH₂NCO); 6.92-6.94 (dd, J=2.1 Hz, J=7.5 Hz, 1H, phenyl); 6.98-7.01 (brt, J=3,0 Hz, 1H, NH); 7.33-7.36 (d, J=5.3 Hz, 1H, H₄); 7.39 (m, 1H, H₃); 7.48-7.51 (dd, J=8.7 Hz, J=2.0 Hz, 1H, H₆); 7.80-7.81 (d, J=2.0 Hz; 1H, H7). ¹³C-NMR (CDCl₃, 90 MHz) δ (ppm): 157.9; 156.1; 152.3; 151.1; 141.2; 129.9; 127.8; 123.0; 121.0; 118.2; 113.0; 112.3; 106.6; 109.7; 107.9; 57.9; 55.4; 53.5; 50.5; 39.4; 27.4; 24.3; 21.0. C H N(%): C₂₃H₂₄BrCl₂N₃O₂ Calc.: C 52.57; H 4.67; N 8.03; Found: C 52.63; H 4.67; N 8.03.

### Example 7

### N-4-(4-(2-methoxyphenyl)piperazine-1-yl)butyl-5-cyano-2-benzo[b]furanylcarbamide

0.37 mmol (141 mg) of HATU and 0.37 mmol (69 mg) of the 5-cyano-2-benzo[b]furanylcarboxylic acid (Dann, O. Liebigs Ann. Chem. 1986, 438-455) are dissolved in 1 ml of DMF at 0° C. and 0,74 mmol (0,13 ml) of DIEA are added. Then 0.33 mmol (87 mg) of 4-(4-(2-methoxyphenyl)piperazine-1-yl)butylamine is dissolved in DMF and dropped into the reaction solution at 0° C. After one hour, the reaction is taken up in CHCl₃ and washed with NaHCO₃ solution and water. After drying with MgSO₄, the solvent is evaporated and purified by flash chromatography (SiO₂; petroleum ether-ethyl acetate: of 1-1 after ethyl acetate).

Yield: 41 mg (28%)

Smp.: 96° C. MS m/z 432 (M+). IR (NaCl): 3400; 2937; 2815; 2227; 1666; 1594; 1527; 1500; 1294; 1240; 1141; 1118; 1025; 842; 746. IH-NMR (CDCl₃, 360 Mhz) δ (ppm): 1.67-1.74 (m, 4H, CH₂—CH₂); 2.49 (t, J=6.9 Hz, 2H, CH₂N); 2.69 (m, 4H, pip); 3.13 (m, 4H, pip); 3.56-3.60 (m, 2H, CH₂NCO); 3.86 (s, 3H, OCH₃); 6.84-7.02 (m, 4H, phenyl); 7.12-7.15 (brt, J=5.1 Hz, 1H, NH); 7.50-7.51 (m, 1H, H₄); 7.55-7.57 (m, 1H, H₃); 7.65-7.68 (m, 1H, H₆); 8.03-8.04 (m, 1H, H₇). ¹³C-NMR (CDCl₃, 90 MHz) δ (ppm): 157.9; 156.1; 152.3; 151.1; 141.2; 129.9; 127.8; 123.0; 121.0; 118.2; 113.0; 112.3; 106.6; 109.7; 107.9; 57.9; 55.4; 53.5; 50.5; 39.4; 27.4; 24.3; 21.0.

### Example 8

### N-4-(4-(2-methoxyphenyl)piperazine-1-yl)butyl-2-benzo[b]tellurophenylcarbamide

Synthesis Analogous to Example 7.

Yield: 73 mg (58%)

Smp.: 85° C. MS m/z 521 (M⁺). IR (KBr): 3320; 3047; 2933; 2815; 1616; 1566; 1541; 1375; 1498; 1240; 1023; 748. ¹H-NMR (CDCl₃, 360 MHz) δ (ppm): 1.62-1.66 (m, 4H, CH₂-CH₂); 2.42-2.45 (t, J=6.7 Hz, 2H, CH₂N); 2.63 (m, 4H, pip); 3.04 (m, 4H, pip); 3.38-3.43 (m, 2H, CH₂NCO); 3.78 (s, 3H, OCH₃); 6.70-6.76 (brt, J=4.3 Hz, 1H, NH); 6.77-6.83 (m, 3H, phenyl); 6.94-6.96 (m, 1H, phenyl); 7.09-7.14 (m, 1H, H₆); 7.28-7.32 (m, 1 H, H₅); 7.74-7.76 (d, J=7,5 Hz, 1H, H₄); 7.84-7.86 (d, J =7.8 Hz, 1H, H₇); 8.13 (s, 1H, H₃). ¹³C-NMR (CDCl₃, 90 MHz) δ (ppm): 166. 1; 152.3; 147.9; 141.2; 140.2; 134.8; 132.3; 129.3; 125.8; 125.5; 122.9; 121.0; 118.2; 111.2; 57.9; 55.3; 53.3; 50.5; 40.4; 27.4; 24.4.

### Example 9

### N-4-(4-(2, 3-dichlorophenyl)piperazine-1 -yl)butyl-2-benzo[b]tellurophenylcarbamide

Synthesis Analogous to Example 7.

Yield: 92 mg (45%)

Smp.: 92° C. MS mn/z 560 (M+). IR (NaCl) : 3288; 2938; 2819; 1651; 1578; 1557; 1448; 1242; 1044; 734. ¹H-NMR (CDCl₃, 360 MHz) δ (ppm): 1.63-1.74 (m, 4H, CH₂—CH₂); 2.48-2.52 (t, J=7.1 Hz, 2H, CH₂N); 2.66 (m, 4H, pip); 3.05 (m, 4 H, pip); 3.46-3.51 (m, 2H, CH₂NCO); 6.70-6.72 (brt, J=4.8 Hz, 1H, NH); 6.83-6.86 (dd, J=1.8 Hz, J=7.8 Hz, 1H, phenyl); 7.07-7.22 (m, 3H, phenyl, H₆); 7.36-7.41 (m, 1H, H₅); 7.81-7.83 (d, J=7.6 Hz, 1H, H₄); 7.91-7.94 (d, J=7.6 Hz, 1H, H₇); 8.17 (m, 1H, H₃). ¹³C-NMR (CDCl₃, 90 MHz) δ (ppm): 166.1; 151.1; 147.9; 140.2; 135.0; 134.9; 134.0; 132.3; 129.2; 127.5; 127.4; 125.8; 125.5; 124.6; 118.6; 58.0; 53.3; 51.2; 40.4; 27.5; 24.4.

### Example 10

### N-4-(4-(2, 3-dichlorophenyl)piperazine-1-yl)butyl-2-indolylcarbamide

Synthesis Analogous to Example 1.

Yield: 48 mg (58% yield in two reaction steps)

Smp.: 148° C. MS m/z 444 (M⁺). IR (NaCl): 3258; 3059; 2938; 2821; 1636; 1577; 1555; 1507; 1448; 1420; 1308; 1241; 1139; 1044; 961; 908; 779; 747; 733; 669; 647. ¹H-NMR (CDCl₃, 360 MHz) δ (ppm): 1.67-1.74 (m, 4H, CH₂—CH₂); 2.47-2.51 (t, J=6.9 Hz, 2H, CH₂N); 2.67 (m, 4H, pip); 3.13 (m, 4H, pip); 3.53-3.55 (m, 2H, CH₂NCO); 6.59-6.66 (brt, J=4.3 Hz, 1H, NHCO); 6.85 (s, 1H, H₃); 6.90-6.93 (m, 1H, phenyl); 7.07-7.17 (m, 3H, phenyl, H₅); 7.28-7.30 (m, 1H, H₆); 7.43-7.46 (m, 1H, H₇); 7.62-7.65 (m, 1H, H₄); 9.56 (s, 1H, NH). ¹³C-NMR (CDCl₃, 90 MHz) δ (ppm): 161.7; 160.4; 151.1; 136.2; 134.0; 130.9; 127.5; 127.4; 121.8; 120.8; 118.5; 111.9; 101.8; 68.2; 57.9; 53.3; 51.1; 39.5; 27.5; 24.3.

### Example 11

### N-4-(4-(2-methoxyphenyl)piperazine-1-yl)butyl-5-cyan-2-indolylcarbamide

Synthesis Analogous to Example 7.

Yield: 109 mg (42%)

Smp.: 170° C. MS m/z 431 (M^{+).} ¹H-NMR (CDCl₃, 360 MHz) δ (ppm): 1.74-1.78 (m, 4H, CH₂—CH₂); 2.54-2.65 (t, J=6.7 Hz, 2H; CH₂N); 2.79 (m, 4H, pip); 3.17 (m, 4H, pip); 3.55-3.59 (m, 2H, CH₂NCO); 3.85 (s, 3H, OCH₃); 6.84-6.87 (d, J=8.5 Hz, 1H, H₅); 6.88-6.90 (m, 3H, phenyl); 6.99-7.05 (m, 2H, phenyl, H₄); 7.07-7.12 (brt, J=3.9 Hz, 1H, NHCO); 7.47-7.50 (dd, J=1.4 Hz, J=8.5 Hz, 1H, H₆); 7.52-7.54 (d, J=8.5 Hz, 1H, H₇); 8.01 (s, 1H, H₃); 10.14 (s, 1H, NH). ¹³C-NMR (CDCl₃, 90 MHz) δ (ppm): 152.2; 140.7; 137.7; 133.3; 127.8; 127.3; 126.7; 123.4; 121.1; 120.2; 118.3; 111.3; 103.9; 102.9; 57.6; 55.4; 53.5; 50.0; 39.2; 27.0; 24.2.

### Example 12

### N-4-(4-(2-methoxyphenyl)piperazine-1-yl)butyl-5-bromo-2-indolylcarbamide

Synthesis Analogous to Example 7.

Yield: 112 mg (60%)

Smp.: 188° C. ¹H-NMR (CDCl₃, 360 MHz) δ (ppm): 1.64-1.74 (m, 4H, CH₂—CH₂); 2.46-2.51 (t, J=7.1 Hz, 2H, CH₂N); 2.68 (m, 4H, pip); 3.12 (m, 4H, pip); 3.49-3.64 (m, 2H, CH₂NCO); 3.83 (s, 3H, OCH₃); 6.68-6.71 (brt, J=5.3 Hz, 1H, NHCO); 6.76-6.77 (d, J=1.8 Hz, 1H, H₄); 6.85-6.87 (d, J=7.8 Hz, 1H, phenyl); 6.92-6.93 (d, J=3.9 Hz, 2H, phenyl); 6.98-7.03 (m, 1H, phenyl); 7.31-7.38 (m, 2H, H₆, H₇); 7.76-7.77 (m, 1H, H₃); 9.64 (s, 1H, NH).

### Example 13

### N-4-(4-(2-methoxyphenyl)piperazine-1-yl)butyl-6-cyan-2-indolylcarbamide

Synthesis Analogous to Example 1.

Yield: 112 mg (58% yield in 2 reaction steps)

Smp.: 174° C. MS m/z 431 (M⁺). IR (NaCl): 2940; 2909; 2803; 2753; 2216; 1645; 1548; 1498; 1321; 1237; 1148; 820; 754; 742. ¹H-NMR (CDCl₃, 360 MHz) δ (ppm): 1.67-1.82 (m, 4H, CH₂-CH₂); 2.47-2.51 (t, J=6.7 Hz, 2H, CH₂N); 2.66 (m, 4H, pip); 3.68-3.70 (m, 2H, CH₂NCO); 3.85 (s, 3H, OCH₃); 3.09 (m, 4H. pip); 6.84-6.94 (m, 4H, phenyl, H₃); 6.99-7.02 (m, 1H, phenyl); 7.13-7.16 (brt, J=5.5 Hz, 1H, NHCO); 7.31-7.34 (m, 1H, H₅); 7.67-7.69 (d, J=8.5 Hz, H₄); 7.84 (s, 1H, H₇); 11.22 (s, 1H, NH). ¹³C-NMR (CDCl₃, 90 MHz) δ (ppm): 161.2; 152.2; 141.1; 135.2; 134.3; 130.5; 123.0; 122.9; 122.7; 120.9; 120.2; 118.1; 117.4; 111.2; 106.6; 102.2; 57.8; 55.3; 53.8; 53.4; 50.4; 39.8; 30.1; 27.3; 24.3. CHN (%): C₂₅H₂₉N₅O₂-1.4 H₂O; Calc.: C, 65.74; H, 7.02; N, 15.33; Found: C, 65.98; H, 7.30; N, 14.87.

### Example 14

### N-4-(4-(2, 3-dichlorophenyl)piperazine-1-yl)butyl-5-brom-2-indolylcarbamide

0,24 mmol (58 mg) of HATU, 0,24 mmol of HOAt (33 mg) and 0,24 mmol (69 mg) of the 5-bromine-2-indolcarboxylic acid are dissolved in 5 ml of DMF at 0° C. in 5 ml of DMF and 0,48 mmol (0,094 ml) DIEA are added. Then 0.26 mmol (78 mg) of 4-(4-(2,3-dichlorophenyl)piperazine-1-yl)butylamine are dissolved in DMF and dropped into the reaction solution at 0° C. After three hours, the reaction is taken up in CHCl₃ and washed with NaHCO₃ solution and water. After drying with MgSO_{4,} the solvent is evaporated and purified by flash chromatography (SiO₂; CHCl₃:MM, 98:2). Yield: 94 mg (74%) MS m/z 524 (M⁺). IR (NaCl): 3234; 2932, 2821; 1637; 1577; 1545; 1282; 1046; 733. ¹H-NMR (CDCl₃, 360 MHz) δ (ppm): 1.57-1.734 (m, 4H, CH₂—CH₂); 2.51 (t, J=6.9 Hz, 2H, CH₂N); 2.66 (m, 4H, pip); 3.07 (m, 4H, pip); 3.51-3.63 (m, 2H, CH₂NCO); 6.64 (brt, J=5.3 Hz, 1H, NHCO); 6.77 (d, J=1.8 Hz, 1H, phenyl, H₄); 6.90 (dd, J=2.1 Hz, J=7.5 Hz. 1H, phenyl); 7.10-7.17 (m, 2H, phenyl); 7.31-7.38 (m, 2H. H₆. H₃); 7,76-7,77 (m, 1H, H₇); 9.68 (s, 1H, NH).

### Example 15

### N-4-(4-(2,3-dichlorophenyl)piperazine-1-yl)butyl-6-cyano-2-indolylcarbamide

Synthesis analogous to example 7.

Yield: 102 mg (59%)

Smp.: 174° C. MS m/z 470(M⁺). IR (NaCl): 3215; 2926; 2821; 1634; 1570; 1506; 1239; 1034; 734. ¹H-NMR (CDCl₃, 360 MHz) δ (ppm): 1.69-1.82 (m, 4H, CH₂—CH₂); 2.51 (t, J=6.9 Hz, 2H, CH₂N); 2.66 (m, 4H, pip); 3.06 (m, 4H, pip); 3.58-3.63 (m, 2H, CH₂NCO); 6.85 (brt, J=5.5 Hz, 1H, NHCO); 6.88-6.91 (m, 2H, phenyl, H₃); 7.09-7.17 (m, 2H, phenyl); 7.35 (dd, J=1.4 Hz, J=8.2 Hz, 1H, H₅); 7.70 (d, J=8.5 Hz, H4); 7.84 (s, 1H, H₇); 10.65 (s, 1 H, NH).

### Example 16

### N-4-(4-(2-methoxyphenyl)piperazine-1 -yl)butyl-2-ferrocenylcarbamide

Synthesis analogous to example 7.

Yield: 155 mg (71%)

MS m/z 475 (M⁺). IR (NaCl): 3318, 2939, 2816, 1630, 1543, 1500, 1241, 1027, 748. ¹H-NMR (CDCl₃, 360 MHz) δ (ppm): 1.66-1.69 (m, 4H, CH₂- CH₂); 2.57 (t, J=6.7 Hz, 2H, CH₂N); 2.77 (m, 4H, pip); 3.16 (m, 4H, pip); 3.39-3.44 (m, 2H, CH₂NCO); 3.86 (s, 3H, OCH₃); 4.17-4.21 (m, 3H, Ferr); 4.32-4.33 (m, 2H, Ferr); 4.68-4.69 (m, 2H, Ferr); 6.02 (brt, J=5.3 Hz, 1H, NH); 6.85-6.94 (m, 3H, phenyl); 6.99-7.03 (m, 1H, phenyl). ¹³C-NMR (CDCl₃, 90 MHz) δ (ppm): 170.3; 152.2; 140.9; 123.1; 120.9; 118.3; 111.2; 77.2; 76.3; 70.3; 69.7; 68.1; 57.9; 55.3; 53.4; 50.1; 39.2; 27.7; 23.8.

### Example 17

### N-4-(4-(2, 3-dichlorophenyl)piperazine-1 -yl)butyl-2-ferrocenylcarbamide

Synthesis analogous to example 7.

Yield: 85 mg (69%)

MS m/z 514 (M⁺). ¹H-NMR (CDCl₃, 360 MHz) δ (ppm): 1.63-1.66 (m, 4H, CH₂—CH₂); 2.48 (t, J=6.9 Hz, 2H, CH₂N); 2.65 (m, 4H, pip); 3.08 (m, 4H, pip); 3.39-3.44 (m, 2H, CH₂NCO); 4.17-4.21 (m, 3H, Ferr); 4.33-4.34 (m, 2H, Ferr); 4.64-4.65 (m, 2H, Ferr); 5.38 (brt, J=5.1 Hz, 1H, NH); 6.92-6.98 (m, 1H, phenyl); 7.11-7.17 (m, 2H, phenyl).

### Example 18

### N-4-(4-(2-methoxyphenyl)piperazine-1-yl)butyl-5-cyano-2-benzo[b]thiophenylcarbamide

0.012 mmol (94 mg) of HATU and 0,012 mmol (25 mg) of the 5-cyano-2-benzo[b]thiophenelcarbamide carboxylic acid (Bridges, A. J. Tetr. Lett. 1992, 7499-7502) are dissolved in 1 ml of DMF and 4 ml of CH₂Cl₂ at 0° C. and 0,024 mmol (0,06 ml) of DIEA added. Then 0.013 mmol (34 mg) of 4-(4-(2-methoxyphenyl) piperazine-1-yl)butylamine are dissolved in CH₂Cl₂ and dropped into the reaction solution at 0° C. After two hours, the reaction is taken up in CHCl₃ and washed with NaHCO₃ solution and water. After drying with MgSO₄, the solvent is evaporated and purified by flash chromatography (SiO₂; methylene chloride-methanol: 98-2).

Yield: 60 mg (91%)

Smp.: 147° C. MS m/z 448 (M⁺). IR (KBr): 3336; 2929; 2816; 2225; 1635; 1500; 1240; 1028; 750. H-NMR (CDCl₃, 360 MHz) δ (ppm): 1.73-1.77 (m, 4H, CH₂—CH₂); 2.59 (t, J=6.4 Hz, 2H, CH₂N); 2.78 (m, 4H, pip); 3.14 (m, 4H, pip); 3.49-3.53 (m, 2H, CH₂NCO) , 3.85 (s, 3H, OCH₃); 6.84-6.92 (m, 5H, phenyl, NH); 6.99-7.04 (m, 1H, phenyl); 7.60 (dd, J=1.4 Hz, J=8.5 Hz, 1H, H₆); 7.88 (s, 1H, H₃); 7.95 (d, J=8.5 Hz, 1H, H₇); 8.12 (d, J=1.1 Hz. 1H, H₄). ¹³C-NMR (CDCl₃, 90 MHz) δ (ppm): 170.3; 152.2; 140.9; 123.1; 120.9; 118.3; 111.2; 77.2; 76.3; 70.3; 69.7; 68.1; 57.9; 55.3; 53.4; 50.1; 39.2; 27.7; 23.8. C H N (%) C₂₄H₂₅Cl₂N₅O. 1H₂O; Calc.: C, 64.35 H 6.48 N, 12.01 S6.87; Found: C, 64.59 H, 6.13 N, 11.77S6.44.

### Example 19

### N-4-(4-(2, 3-dichlorophenyl)piperazine-1-yl)butyl-5-cyan-2-benzo[b]thiophenylcarbamide

Synthesis Analogous to Example 18.

Yield: 57 mg (96%)

Smp.: 190° C. MS m/z 487 (M⁺). IR (KBr): 3319; 2929; 2819; 2227; 1633; 1560; 1448; 1242; 1045; 755. ¹H-NMR (CDCl₃, 360 MHz) δ (ppm): 1.65-1.76 (m, 4H, CH₂—CH₂); 2.49 (t, J=6.7 Hz, 2H, CH₂N); 2.65 (m, 4H, pip); 3.04 (m, 4H, pip); 3.49-3.55 (m, 2H, CH₂NCO); 6.78 (br, t, J=5.0 Hz, 1H, NH); 6.85 (dd J=1.8 Hz, J=7.8Hz, 1H, phenyl); 7.09-7.17 (m, 2H, phenyl); 7.62 (dd, J=1.4 Hz, J=8.5 Hz, 1H, H₆); 7.77 (s, 1H, H₃); 7.95 (d, J=8.2 Hz, H₇); 8.13 (d, J=1.4 Hz, 1H, H₄). ¹³C-NMR (CDCl₃, 90 MHz) δ (ppm): 161.4; 151.1; 144.5; 142.0; 138.8, 134.1; 129.4, 127.9; 127.5, 127.4; 124.7; 123.9; 123.8; 118.9; 118.4; 108.9; 57.9; 53.3; 51.2; 40.2; 27.4; 24.3. C H N (%) C₂₀H₂₆Cl₂N₄O S.1.46 H₂O Calc.: C, 56.11 H, 5.28 N, 10.90; Found: C, 56.51 H, 5.06 N, 10.45.

### Example 20

### N-4-(4-(2-methoxyphenyl)piperazine-1-yl)butyl-6-cyano-2-benzo[b]thiophenylcarbamide

Synthesis Analogous to Example 18.

Yield: 30 mg (43%)

Smp.: 124° C. MS m/z 448 (M⁺). IR (KBr): 3290; 2937; 2816; 2225; 1619; 1543; 1500; 1242; 1026; 751. ¹H-NMR (CDCl₃, 360 MHz) δ (ppm): 1.72-1.90 (m, 4H, CH₂—CH₂); 2.90 (t, J=7.3 Hz, 2H, CH₂N); 3.15 (m, 4H, pip); 3.31 (m, 4H, pip); 3.50-3.56 (m, 2H, CH₂NCO), 3.82 (s, 3H, OCH₃); 6.84-6.92 (m, 3 H, phenyl); 7.01-7.07 (m, 1H, phenyl); 7.13 (br, t, J=5.7, 1H, NH); 7.53 (dd, J=1.4 Hz, J=8.2 Hz, 1H, H₅); 7.84 (d, J=7.8 Hz, 1H, H₄); 7.84 (s, 1H, H₃); 8.10-8.11 (m, 1H, H₇). ¹³C-NMR (CDCl₃, 90 MHz) δ (ppm): 161.5; 152.3; 141.9; 141.0; 140.4; 127.5; 127.4. 125.6; 124.2; 123.1. 120.9; 118.8; 118.1; 111.3; 109.5; 77.2; 76.3; 70.3; 69.7; 68.1; 57.9; 55.3; 53.4; 50.4; 40.1; 27.3; 24.2.

### Example 21

### N-4-(4-(2, 3-dichlorophenyl)piperazine-1 -yl)butyl-6-cyano-2-benzo[b]thiophenylcarbamide

Synthesis analogous to example 18.

Yield: 26 mg (43%)

Smp.: 137° C. MS m/z 486 (M⁺). IR (KBr): 3335; 2933; 2821; 2226; 1638; 1544; 1448; 1242; 1044; 735. H-NMR (CDCl₃, 360 MHz) δ (ppm): 1.67-1.74 (m, 4H. CH₂—CH₂); 2.49 (t, J=6.4 Hz, 2H, CH₂N); 2.65 (m, 4H, pip); 3.04 (m, 4H, pip); 3.49-3.54 (m, 2H, CH₂NCO); 6.75 (br, t, J=4.1 Hz, 1H, NH); 6.84 (dd, J=1.8 Hz, 7.8 Hz, 1H, phenyl); 7.08-7.17 (m, 2H, phenyl); 7,60-7,62 (m, 1H, H₅); 7.78 (s, 1H, H₃); 7.88-7.90 (m, 1H,H₄); 8.19 (br, s, 1H, H₇). 1³C-NMR (CDCl₃, 90 MHz) o (ppm): 161.4; 151.1; 143.6; 141.8; 140.3; 134.1; 127.5; 127.4; 125.2; 124.7. 124.2; 118.7; 118.4; 109.5; 109.5; 58.0; 53.3; 51.3; 51.2; 40.3; 27.4; 24.3. C H N (%): C₂₄H₂₅Cl₂N₅O-H₂O; Calc.: C, 59.02; H, 5.57; N, 14.34. Found: C, 58.76; H, 5.30; N, 14.19.

### Example 22

### N-4-(4-(2-methoxyphenyl)piperazine-1 -yl)butyl-5-brom-2-benzo[b]thiophenylcarbamide

Synthesis Analogous to Example 7.

Yield: 73 mg (58%)

Smp.: 156° C. MS m/z 502 (M⁺). IR (KBr): 3316; 2934; 2821; 1633; 1558; 1500; 1242; 1026; 750. ¹H-NMR (CDCl₃, 360 MHz) δ (ppm): 1.69-1.72 (m, 4H, CH₂—CH₂); 2.57 (t, J=6.5 Hz, 2H, CH₂N); 2.77 (m, 4H, pip); 3.12 (m, 4H, pip); 3.49-3.51 (m, 2H, CH₂NCO); 3.85 (s, 3 H, OCH₃); 6.83-6.91 (m, 4H, phenyl); 7.01-7.06 (m, 1H, phenyl); 7.15 (brt, J=4.9 Hz, 1H, NH); 7.42 (dd, J=8.5 Hz, J=1.8 Hz, 1H, H₆); 7.60 (d, J=8.5 Hz, 1H, H₇); 7.73 (s, 1H, H₃); 7.86 (d, J=1.7 Hz, 1H, H₄). ¹³C-NMR (CDCl₃, 90 MHz) δ (ppm): 170.3; 152.2; 140.9; 123.1; 120.9; 118.3; 111.2; 77.2; 76.3; 70.3; 69.7; 68.1; 57.9; 55.3; 53.4; 50.1; 39.2; 27.7; 23.8. CHN: C₂₄H₂₈BrN₃O₂S (%): Calc.: C, 57.37 H, 5.62 N, 8.36; Found: C, 65.98 H, 7.30N, 14.87.

### Example 23

### N-4-(4-(2, 3-dichlorophenyl)piperazine-1-yl)butyl-5-brom-2-benzo[b]thiophenylcarbamide

Synthesis Analogous to Example 7.

Yield: 78 mg (60%)

Smp.: 178° C. MS m/z 541 (M⁺). IR (KBr;): 3316; 2929; 2821; 1631; 1560; 1242; 1068; 756. H-NMR (CDCl₃, 360 MHz) δ (ppm): 1.63-1.69 (m, 4H, CH₂—CH₂); 2.48 (t, J=6.7 Hz, 2H, CH₂N); 2.64 (m, 4H, pip); 3.04 (m, 4H, pip); 3.48-3.53 (m, 2H, CH₂NCO); 6.71 (br, t, J=5.1 Hz, 1H, NH); 6.83-6.86 (m, 1H, phenyl); 7.09-7.17 (m, 2H, phenyl); 7.51 (dd, J=1.8 Hz. J=8.5 Hz, 1H, H₆); 7.67 (s. 1H, H₃); 7.72 (d, J=8.5 Hz, 1H, H₇); 7.95 (d, J=1.8 Hz, 1H, H₄). ¹³C-NMR (CDCl₃, 90 MHz) δ (ppm): 161.9; 160.8; 140.7; 140.6. 139.2; 134. 1; 129.3; 127.4; 127.3; 124.6; 124.1; 123.8. 118.9; 118.5; 57.9; 55.3; 51.2; 40.2; 27.5; 24.4. C H N (%): C₂₃H₂₄BrCl₂N₃OS.0.25H₂O; Calc.: C, 50.61 H, 4.52 N, 7.70 S, 5.87; Found: C, 50.61 H, 4.49 N, 7.64 S, 5.87.

### Example 24

### N-4-(4-(2-methoxyphenyl)piperazine-1-yl)butyl-2-indolylcarbamide

Synthesis Analogous to Example 7.

Yield: 89 mg (55%)

MS m/z 406 (M+). IR (NaCl): 3251; 3055; 2935; 2809; 1639; 1549; 1241; 1016; 746. ¹H-NMR (CDCl₃, 360 MHz) δ (ppm): 1.67-1.74 (m, 4H,CH₂—CH₂); 2.48 (t, J=6.7 Hz, 2H, CH₂N); 2.67 (m, 4H, pip); 3.11 (m, 4H, pip); 3.51-3.56 (m, 2H, CH₂NCO); 3.85 (s, 1H, OCH₃); 6.59 (br, t, J=4.9 Hz, 1H, NHCO); 6.83-6.94 (m, 3H, phenyl); 6.97-7.02 (m, 1H, phenyl); 7.11-7.15 (m, 1H, H₅); 7.25 (s, 1H, H₃); 7.29 (dd, J=1.1 Hz, J=7.1 Hz, 1H, H₆); 7.43 (d, J=8.9 Hz, 1H, H₇); 7.63 (d, J=8.9 Hz, 1H, H₄); 9.50 (s, 1H).

### Example 25

### N-4-(4-(2,3-dichlorophenyl)piperazine-1-yl)butyl-5-cyano-2-indolylcarbamide

Synthesis Analogous to Example 7.

Yield: 102 mg (59%)

Smp.: 96° C. MS m/z 470 (M⁺). IR (KBr): 3315; 2926; 2821; 2218, 1634; 1570; 1506; 1239; 1043; 734. H-NMR (CDCl₃, 360 MHz) δ (ppm): 1.69-1.82 (m, 4H, CH₂-CH₂); 2.51 (t, J=6.9 Hz, 2H, CH₂N); 2.66 (m, 4H, pip); 3.06 (m, 4H, pip); 3.58-3.63 (m, 2H, CH₂NCO); 6.85 (br, t,, J=5.5 Hz, 1H, NHCO); 6.88-6.91 (m, 2H, phenyl, H₃); 7.09-7.17 (m, 2H, phenyl); 7.35 (dd, J=1.4 Hz, J=8.2 Hz, 1H, H₅); 7.70 (d, J=8.5 Hz, H₄); 7.84 (s, 1H, H₇); 10.65 (s, 1H, NH). ¹³C-NMR (CDCl₃, 90 MHz) δ (ppm): 161.2; 151.1; 135.2; 134.3; 134.0; 130.5; 127.5; 127.4; 124.6; 123.1; 122.8; 120.1; 118.5; 117.2; 106.9; 102.0; 58.7; 53.3; 51.2; 41.1; 39.9; 24.4. C H N (%): C₂₄H₂₅Cl₂N₅O.H₂O; Calc.: C, 59.02; H, 5.57; N, 14.34. Found: C, 58.76; H, 5.30; N, 14.19.

Biological Activity

The biological activities of the compounds of the invention were determined in radio ligand binding assays. All radio ligand experiments were carried out in accordance with methods described by us (Hübner, H. et al. J. Med. Chem. 2000, 43, 756-762). For the measurement of the affinities to the receptors of the D2 family, membrane homogenates of ovarian cells of the Chinese hamsters (CHO cells) were used each of which stably expressed the human D2long-, the human D2short- (Hayes, G. et al. Mol. Endocrinol. 1992, 6, 920-926), the human D3- (Sokoloff, P. et al. Eur. J. Pharmacol. 1992, 225, 331-337) or the human D4.4-receptor subtype (Asghari, V. J. Neurochem. 1995, 65, 1157-1165). In general, the binding assays were carried out by incubation of the receptor homogenates with the radio ligand [³H]Spiperon and the compound to be tested in different concentrations. The affinities to the D1 receptor were determined with native membrane homogenates obtained from the striatum of a pig and with the D1-selective radio ligand [³H]SCH 23390.

For the purpose of determining the binding strengths of the compounds to the serotonin receptor subtypes 5-HT1A and 5-HT2 cortex membrane preparations of a pig were incubated with the radio ligands [³H]8-OH-DPAT (for 5-HT1A) or [³H]Ketanserin (5-HT2) and the compounds. Indications of a simultaneous binding of the compounds to the serotonergic 5-HT2 receptor and to the adrenergic receptor subtype a1 upon labelling with the radio ligand [³H]Ketanserin were confirmed in a parallel experiment with selective blocking of the a1 receptor by Prazosin. Therefore Ki values determined in the presence of 10 μM of Prazosin represent the sole bond to the 5-HT2 receptor. In addition, the affinity to α1 receptors of the pig were determined in a separate experiment with the α1-selective radio ligand [³H]Prazosin.

The results of the receptor binding assays on the dopamine receptor subtypes are summarised in table 1.

All of the compounds tested in the binding assay showed good to very good affinities to the dopamine receptors with a clear binding preference to the subtypes of the D2 family. Independently of the partial structure, a clear selectivity to the D3 receptor is always evident. The highest D3 affinity may be achieved if benzo[b]thiophene or indol is used as the heteroarene component. For example, compounds of the examples 1, 2, 10 to 13 and 19 to 22 have excellent Ki values between 0.23 and 0.57 nM.

The substitution pattern of the arylpiperazine component primarily influences the degree of selectivity of the D3 affinity vis-à-vis the other receptor subtypes. With selection coefficients of more than 1000, the 2,3-dichlorophenyl-substituted compounds (examples 2, 6, and 10) display a D3 selectivity hitherto not described with concomitant subnanomolar affinity. It is interesting that the ferrocenyl derivatives of the examples 16 and 17 are characterised by a high D4 affinity, example 17 with Ki values of 0.47 nM for the D3 receptor and 0.63 nM for the D4 receptor showing an extraordinary receptor binding profile.

Tests to determine the intrinsic activity of the compounds of the examples were carried out in a mitogenesis assay as described in literature (Hübner, H. et al. J. Med. Chem. 2000, 43, 4563-4569; Löber, S. Bioorg. Med. Chem. Lett. 2002, 12.17, 2377-2380). A partial agonistic activity of 49% of the maximum receptor stimulation is illustrative for the compound of example 1, which may be triggered by the full agonist Quinpirol as the reference compound. Curve calculations of this concentration-activity test resulted in an EC₅₀ value of 0.38 nM.

TABLE 1
Binding data and selectivity patterns of the compounds of the
formulae (I) to (IV) for the dopamine receptors pD1, hD2long, hD2short, hD3
and hD4.4

| | Ki-values in [nM]^{a} | | | | | D3 selectivity | | |
|---|---|---|---|---|---|---|---|---|
| Compound | D1 | D2long | D2short | D3 | D4.4 | D2long/D3 | D2short/D3 | D4.4/D3 |
| Example 1 | 670 | 87 | 52 | 0.23 | 15 | 380 | 230 | 65 |
| Example 2 | 8800 | 3300 | 2600 | 0.5 | 340 | 6600 | 5200 | 680 |
| Example 3 | 1100 | 110 | 84 | 1.1 | 30 | 100 | 76 | 27 |
| Example 4 | 2900 | 320 | 80 | 1.2 | 93 | 270 | 67 | 78 |
| Example 5 | 590 | 96 | 61 | 0.69 | 17 | 140 | 88 | 25 |
| Example 6 | 2100 | 10000 | 4800 | 3.4 | 3100 | 2900 | 1400 | 910 |
| Example 7 | 1400 | 130 | 89 | 4.2 | 57 | 31 | 21 | 14 |
| Example 8 | 380 | 63 | 39 | 0.72 | 35 | 88 | 54 | 49 |
| Example 9 | 1400 | 91 | 48 | 0.55 | 150 | 170 | 87 | 270 |
| Example | 1100 | 3100 | 1600 | 0.56 | 1700 | 5500 | 2900 | 3000 |
| Example | 920 | 140 | 99 | 0.57 | 24 | 250 | 180 | 44 |
| Example | 390 | 110 | 44 | 0.24 | 16 | 460 | 180 | 67 |
| Example | 460 | 160 | 100 | 0.25 | 40 | 640 | 400 | 160 |
| Example | 4200 | 2300 | 770 | 0.73 | 600 | 3200 | 1100 | 820 |
| Example | 1700 | 340 | 110 | 0.35 | 630 | 970 | 310 | 1800 |
| Example | 1500 | 110 | 78 | 6.5 | 0.40 | 17 | 12 | 0.061 |
| Example | 630 | 31 | 19 | 0.47 | 0.63 | 66 | 40 | 1.3 |
| Example | 430 | 68 | 39 | 0.46 | 45 | 150 | 85 | 98 |
| Example | 1700 | 410 | 310 | 0.25 | 650 | 1600 | 1200 | 2600 |
| Example | 1100 | 210 | 130 | 0.33 | 37 | 640 | 390 | 110 |
| Example | 1700 | 180 | 60 | 0.26 | 72 | 690 | 230 | 280 |
| Example | 550 | 49 | 30 | 0.26 | 58 | 190 | 120 | 220 |
| Example | 4700 | 1700 | 970 | 3.2 | 1700 | 1500 | 300 | 530 |
| Example | 1200 | 200 | 160 | 0.70 | 40 | 290 | 230 | 57 |
| Example | 1700 | 140 | 27 | 0.91 | 210 | 150 | 30 | 230 |

^{a}Average values from 2-9 individual experiments carried out as triplicates

The investigation of the affinities to the serotonin receptor subtypes 5-HT1A and 5-HT2 and to the adrenergic receptor al is described in table 2. Irrespective of the partial structures of the derivatives, a preferred binding to the 5-HTIA subtype when compared with 5-HT2 can be observed. With measured Ki values of 8 to 19 nM, the compounds of the examples 1, 3, 7, 8 and 16 are characterised by a high affinity to the α1 receptor.

Considerations concerning structure/activity show a clear dependence on the substitution pattern of the aryl piperazine partial structure for the binding to these receptors. As with the dopamine receptors, the binding to the 5-HT and to the α1receptor is clearly diminished in the derivatives with a 2,3-dichlorophenyl radical which results in an extension of the selectivity spectrum vis-à-vis the D3 receptor affinity of these compounds.

TABLE 2
Binding data of substances of the formulae (I) to (IV) for the
serotonin receptors p5-HT1A, p5-HT2 and for the adrenergic
receptor subtype pα1

Ki values in [nM]^{a}

| Compounds | 5-HT1A | 5-HT2^{b} | α1^{c} | α1^{d} |
|---|---|---|---|---|
| Example 1 | 41 | 350 | 15 | 6.4 |
| Example 2 | 360 | 2000 | — | 370 |
| Example 3 | 17 | 660 | 14 | 3.3 |
| Example 4 | 480 | 11000 | — | 160 |
| Example 5 | 68 | 140 | — | 5.3 |
| Example 6 | 2500 | 540 | — | 1300 |
| Example 7 | 37 | 390 | 8.2 | 11 |
| Example 8 | 69 | 420 | 15 | 3.5 |
| Example 9 | 130 | 730 | — | 100 |
| Example 10 | 610 | 1700 | — | 220 |
| Example 11 | 83 | 440 | 24 | 5.9 |
| Example 12 | 440 | 280 | — | 6.4 |
| Example 13 | 47 | 220 | — | 4.3 |
| Example 14 | 1600 | 690 | — | 500 |
| Example 15 | 390 | 320 | — | 2000 |
| Example 16 | 0.60 | 500 | 19 | 30 |
| Example 17 | 27 | 250 | — | 73 |
| Example 18 | 54 | 580 | — | 2.9 |
| Example 19 | 190 | 280 | — | 230 |
| Example 20 | 71 | 660 | — | 8.3 |
| Example 21 | 110 | 290 | — | 45 |
| Example 22 | 180 | 760 | — | 2.5 |
| Example 23 | 430 | 14000 | — | 320 |
| Example 24 | 32 | 420 | 11 | 7.3 |
| Example 25 | 190 | 220 | — | 220 |

^{a}Average values from 2 to 6 individual experiments carried out with triplicates
^{b}Ki value in case of simultaneous incubation with 10 μM of Prazosin
^{c}Ki value derived from the highly affine binding site in case of labelling with [³H]Ketanserin
^{d}Ki value from the competitive experiment with the α1 selective radio ligand [³H]Prazosin

1. A compound of the general formula (I) 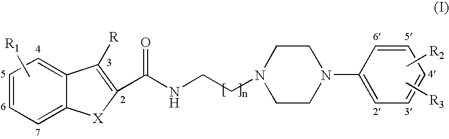
wherein:
n=1-4and
R=hydrogen, alkyl or halogen and
(a) X=S or O:
(i) when R₁ is hydroxy, alkyloxy, alkenyl, alkinyl, aryl, acyl, alkoxycarbonyl or cyano, each of R₂ and R₃ are independently selected from hydrogen, hydroxy, alkyloxy, alkyl, alkenyl, alkinyl, aryl, halogen, trifluoromethyl, acyl, alkoxycarbonyl and cyano,
(ii) when R₁ is hydrogen, alkyl, halogen or trifluoromethyl, R₂ is selected from hydroxy, alkenyl, alkinyl, aryl, acyl, alkoxycarbonyl and cyano and R₃ is selected from hydrogen, hydroxy, alkyloxy, alkyl, alkenyl, alkinyl, aryl, halogen, trifluoromethyl, acyl, alkoxycarbonyl and cyano,
or
(b) X=NH: R₁ is selected from hydrogen, hydroxy, alkyl, alkyloxy, alkenyl, alkinyl, aryl, trifluoromethyl, acyl, alkoxycarbonyl, halogen and cyano and each of R₂ and R₃ are selected independently from hydrogen, hydroxy, alkyloxy, alkyl, alkenyl, alkinyl, aryl, halogen, trifluoromethyl, acyl, alkoxycarbonyl and cyano, with the proviso that the compound is not N-4-(4-(2-methoxyphenyl)piperazine-1-yl)butyl-2-indolylcarbamide,
or
(c) X=Te:
R₁ is selected from hydrogen, hydroxy, alkyl, alkyloxy, alkenyl, alkinyl, aryl, halogen, trifluoromethyl, acyl, alkoxycarbonyl and cyano and each of R₂ and R₃ are selected independently from hydrogen, hydroxy, alkyloxy, alkyl, alkenyl, alkinyl, aryl, halogen, trifluoromethyl, acyl, alkoxycarbonyl and cyano.
wherein the groups alkyl, alkenyl, alkinyl and aryl may optionally be substituted independently of one another,
and pharmaceutically acceptable salts of this compound.

2. A compound according to claim 1 wherein
n=1-4
and
X=Te, when R=hydrogen, alkyl or halogen and R₁ is substituted by the radicals hydrogen, hydroxy, alkyl, alkyloxy, alkenyl, alkinyl, aryl, halogen, trifluoromethyl, acyl, alkoxycarbonyl or cyano and R₂ and R₃ are substituted individually or jointly by the radicals hydrogen, hydroxy, alkyloxy, alkyl, alkenyl, alkinyl, aryl, halogen, trifluoromethyl, acyl, alkoxycarbonyl or cyano,
or
X=S or O, when R=hydrogen, alkyl or halogen and R₁ is substituted by the radicals hydroxy, alkyloxy, alkenyl, alkinyl, aryl, acyl, alkoxycarbonyl or cyano and R₂ and R₃ are substituted individually or jointly by the radicals hydrogen, hydroxy, alkyloxy, alkyl, alkenyl, alkinyl, aryl, halogen, trifluoromethyl, acyl, alkoxycarbonyl or cyano,
or
X=S or O, when R=hydrogen, alkyl or halogen and R₁ is substituted by the radicals hydrogen, alkyl, halogen or trifluoromethyl and R₂ and R₃ are substituted individually or jointly by the radicals hydroxy, alkenyl, alkinyl, aryl, acyl, alkoxycarbonyl or cyano,
or
X=NH, when R=hydrogen, alkyl or halogen and R₁ is substituted by the radicals hydroxy, alkyl, alkyloxy, alkenyl, alkinyl, aryl, trifluoromethyl, acyl, alkoxycarbonyl or cyano, it being required that alkyl and alkyloxy contain at least two carbon atoms, and R₂ and R₃ are substituted individually or jointly by the radicals hydrogen, hydroxy, alkyloxy, alkyl, alkenyl, alkinyl, aryl, halogen, trifluoromethyl, acyl, alkoxycarbonyl or cyano and alkyloxy comprises at least two carbon atoms.

3. A compound according to claim 1 having the general formula (Ia) or (Ib): 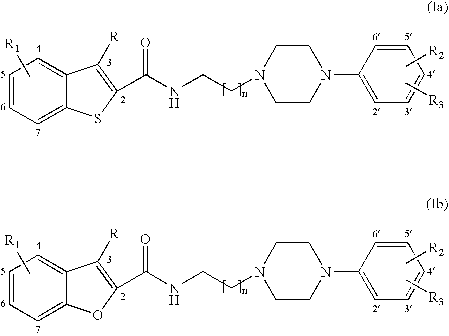
wherein:
n=1-4,
R=hydrogen, C₁-C₆-alkyl or halogen,
when R₁ is hydroxy, C₁-C₆-alkyloxy, C₂-C₆-alkenyl, C₂-C₆-alkinyl, phenyl that may optionally be substituted with a methoxy group or halogen, C₁-C₆-acyl, C₁-C₆-alkoxy carbonyl or cyano, each of R₂ and R₃ are independently selected from hydrogen, hydroxy, C₁-C₆-alkyloxy, C₁-C₆-alkyl, C₂-C₆-alkenyl, C₂-C₆-alkinyl, phenyl that may optionally be substituted with a methoxy group or halogen, halogen, trifluoromethyl, C₁-C₆-acyl, C₁-C₆-alkoxycarbonyl and cyano,
when R₁ is hydrogen, C₁-C₆-alkyl, halogen or trifluoromethyl, R₂ is selected from hydroxy, C₂-C₆-alkenyl, C₂-C₆-alkinyl, phenyl that may optionally be substituted with a methoxy group or halogen, C₁-C₆-acyl, C₁-C₆-alkoxycarbonyl and cyano, and R₃ is selected from hydrogen, hydroxy, C₁-C₆-alkyl, C₁-C₆-alkyloxy, C₂-C₆-alkenyl, C₂-C₆-alkinyl, phenyl that may optionally be substituted with a methoxy group or halogen, halogen, trifluoromethyl, C₁-C₆-acyl, C₁-C₆ alkoxycarbonyl and cyano,
wherein the groups C₁-C₆-alkyl, C₂-C₆-alkenyl and C₂-C₆-alkinyl may optionally also be substituted independently of one another,
and pharmaceutically acceptable salts thereof.

4. A compound according to claim 1 of the general formula (Ic): 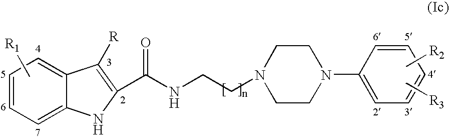
wherein:
n=1-4,
R=hydrogen, C₁-C₆-alkyl or halogen,
R₁ is selected from hydrogen, hydroxy, C₁-C₆-alkyl, C₁-C₆-alkoxy, C₂-C₆-alkenyl, C₂-C₆-alkinyl, phenyl that may optionally be substituted with a methoxy group or halogen, trifluoromethyl, C₁-C₆-acyl, C₁-C₆-alkoxycarbonyl, fluorine, chlorine, bromine and cyano,
each of R₂ and R₃ are independently selected from hydrogen, hydroxy, C₁-C₆-alkyl, C₁-C₆-alkyloxy, C₂-C₆-alkenyl, C₂-C₆-alkinyl, phenyl that may optionally be substituted with a methoxy group or halogen, halogen, trifluoromethyl, C₁-C₆-acyl, C₁-C₆-alkoxycarbonyl and cyano,
wherein the groups C₁-C₆ alkyl, C₂-C₆ alkenyl and C₂-C₆ alkinyl may optionally also be substituted independently of one another,
and pharmaceutically acceptable salts of this compound, with the proviso that the compound is not N-4-(4-(2-methoxyphenyl)piperazine-1-yl)butyl-2-indolylcarbamide.

5. A compound according to claim 4, wherein
(a) when R₁ is hydroxy, C₂-C₆-alkenyl, C₂-C₆-alkinyl, phenyl that may optionally be substituted with a methoxy group or halogen, trifluoromethyl, C₁-C₆-acyl, C₁-C₆-alkoxycarbonyl or cyano, each of R₂ and R₃ are independently selected from hydrogen, hydroxy, C₁-C₆-alkyl, C₁-C₆-alkyloxy, C₂-C₆-alkenyl, C₂-C₆-alkinyl, phenyl that may optionally be substituted with a methoxy group or halogen, halogen, trifluoromethyl, C₁-C₆-acyl, C₁-C₆-alkoxycarbonyl and cyano,
and
(b) when R₁ is hydrogen, C₁-C₆-alkyl, C₁-C₆-alkyloxy or halogen, R₂ is selected from hydroxy, C₂-C₆-alkenyl, C₂-C₆-alkinyl, phenyl that may optionally be substituted with a methoxy group or halogen, C₁-C₆-acyl, C₁-C₆-alkoxycarbonyl and cyano, and R₃ is selected from hydrogen, hydroxy, C₁-C₆-alkyl, C₁-C₆-alkyloxy, C₂-C₆-alkenyl, C₂-C₆-alkinyl, phenyl that may optionally be substituted with a methoxy group or halogen, halogen, trifluoromethyl, C₁-C ₆-acyl, C₁-C ₆-alkoxycarbonyl and cyano,
wherein the groups C₁-C₆ alkyl, C₂-C₆ alkenyl and C₂-C₆ alkinyl may optionally also be substituted independently of one another,
and pharmaceutically acceptable salts of this compound.

6. A compound according to claim 1 wherein
the substituent R₁ is in position 5 or 6 of the heterocycle, and
the substituents R₂ and R₃ are in the positions 2 or 3, respectively, or in the positions 2 or 4, respectively, of the phenyl ring; the respective other substituent being in position 2 of the phenyl ring in the event that one of the two substituents R₂ and R₃ is a hydrogen atom.

7. A compound according to claim 1 wherein n=3.

8. A compound of the general formula (IV): 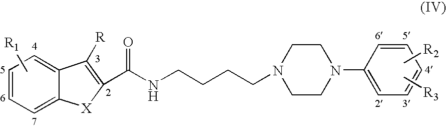
wherein:
X=S,NH or O,
R is selected from hydrogen, C₁-C₆-alkyl, fluorine, chlorine and bromine,
R₁ is selected from hydrogen, C₁-C₆-alkoxy, C₁-C₆-alkyl, fluorine, chlorine, bromine, trifluoromethyl and cyano, R₁ being in position 5 or δ of the heterocycle,
R₂ and R₃ are independently selected from hydrogen, C₁-C₆-alkyloxy, C₁-C₆-alkyl, fluorine, chlorine, bromine and trifluoromethyl, R₂ and R₃ being in the positions 2 or 3, respectively, or in the positions 2 or 4, respectively, of the phenyl ring, and the respective other substituent being in position 2 of the phenyl ring in the event that one of the two substituents R₂ and R₃ is a hydrogen atom
wherein the C₁-C₆ alkyl groups are optionally substituted independently of one another
and pharmaceutically acceptable salts of this compound with the proviso that the compound is not N-4-(4-(2-methoxyphenyl)piperazine-1-yl)butyl-2-indolylcarbamide.

9. A compound according to claim 8, wherein
when X=NH, then R₁ is selected from hydrogen, C₁-C₃-alkyloxy, C₁-C₃-alkyl, fluorine, chlorine, bromine and cyano,
and
when X=S or O, then R₁ is selected from hydrogen, C₁-C₃-alkyl, fluorine, chlorine, bromine, cyano and trifluoromethyl.

10. A compound according to claim 1 selected from
N-4-(4-(2-methoxyphenyl)piperazine-1-yl)butyl-5-cyano-2-benzo[b]thiophenylcarbamide,
N-4-(4-(2,3-dichlorophenyl)piperazine-1-yl)butyl-5-cyano-2-benzo[b]thiophenylcarbamide,
N-4-(4-(2-methoxyphenyl)piperazine-1-yl)butyl-6-cyano-2-benzo[b]thiophenylcarbamide,
N-4-(4-(2,3-dichlorophenyl)piperazine-1-yl)butyl-6-cyano-2-benzo[b]thiophenylcarbamide,
N-4-(4-(2-methoxyphenyl)piperazine-1-yl)butyl-2-benzo[b]thiophenylcarbamide,
N-4-(4-(2,3-dichlorophenyl)piperazine-1-yl)butyl-2-benzo[b]thiophenylcarbamide,
N-4-(4-(2-methoxyphenyl)piperazine-1-yl)butyl-5-bromo-2-benzo[b]thiophenylcarbamide,
N-4-(4-(2,3-dichlorhenyl)piperazine-1-yl)butyl-5-bromo-2-benzo[b]thiophenylcarbamide,
N-4-(4-(2,3-dichlorophenyl)piperazine-1-yl)butyl-2-indolylcarbamide,
N-4-(4-(2-methoxyphenyl)piperazine-1-yl)butyl-5-cyano-2-indolylcarbamide,
N-4-(4-(2-methoxyphenyl)piperazine-1-yl)butyl-5-bromo-2-indolylcarbamide,
N-4-(4-(2-methoxyphenyl)piperazine-1-yl)butyl-6-cyano-2-indolylcarbamide,
N-4-(4-(2,3-dichlorophenyl)piperazine-1-yl)butyl-5-bromo-2-indolylcarbamide,
N-4-(4-(2,3-dichlorophenyl)piperazine-1-yl)butyl-6-cyano-2-indolylcarbamide,
N-4-(4-(2,3-dichlorophenyl)piperazine-1-yl)butyl-5-cyano-2-indolylcarbamide,
N-4-(4-(2-methoxyphenyl)piperazine-1-yl)butyl-5-cyano-2-benzo[b]furanylcarbamide,
N-4-(4-(2-methoxyphenyl)piperazine-1-yl)butyl-2-benzo[b]furanylcarbamide,
N-4-(4-(2,3-dichlorophenyl)piperazine-1-yl)butyl-2-benzo[b]furanylcarbamide,
N-4-(4-(2-methoxyphenyl)piperazine-1-yl)butyl-5-bromo-benzo[b]furanylcarbamide,
N-4-(4-(2,3-dichlorophenyl)piperazine-1-yl)butyl-5-bromo-2-benzo[b]furanylcarbamide,
N-4-(4-(2-methoxyphenyl)piperazine-1-yl)butyl-2-benzo[b]tellurophenylcarbamide und
N-4-(4-(2,3-dichlorophenyl)piperazine-1-yl)butyl-2-benzo[b]tellurophenylcarbamide
and pharmaceutically acceptable salts thereof.

11. A compound of the general formula (II) 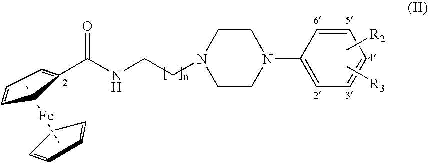
wherein
n=1-4 and R₁ and R₂ individually or jointly represent the radicals hydrogen, hydroxy, alkyloxy, alkyl, alkenyl, alkinyl, aryl, halogen, trifluoromethyl, acyl, alkoxycarbonyl or cyano.

12. A compound according to claim 11 wherein each of R₁ and R₂ is independently selected from hydrogen, hydroxy, C₁-C₆ alkyloxy, C₁-C₆ alkyl, C₂-C₆ alkenyl, C₂-C₆ alkinyl, aryl, fluorine, chlorine, bromine, trifluoromethyl, C₁-C₆ acyl, C₁-C₆ alkoxycarbonyl and cyano wherein the groups C₁-C₆ alkyl, C₂-C₆ alkenyl, C₂-C₆ alkinyl and aryl may optionally also be substituted independently of one another.

13. A compound according to claim 12 selected from
N-4-(4-(2-methoxyphenyl)piperazine-1-yl)butyl-2-ferrocenylcarbamide and
N-4-(4-(2,3-Dichlorophenyl)piperazine-1-yl)butyl-2-ferrocenylcarbamide.

14. A therapeutic agent containing one or more of the compounds according to claim 1.

15. A therapeutic agent according to claim 14 which additionally contains L-DOPA for simultaneous or sequential administration to the patient.

16. The use of a compound according to claim 1 for preparing a therapeutic agent for the therapy or prevention of cocaine, alcohol, opiate and nicotine addiction; neurodegenerative disorders, especially Parkinson's disease; sexual dysfunction; depression or schizophrenia.

17. The use of a compound according claim 1 for preparing a therapeutic agent for the therapy or prevention of hyperprolactinaemia; hyperprolactinoma; glaucoma; cognitive disorders; restless leg syndrome; hyperactivity syndrome (ADHS); locomotion disorders associated with Parkinson's disease; L-DOPA-induced disorders, Segawa syndrome; tardive locomotion disorders as well as for medication-assisted ablactation after pregnancies.

18. The use according to claim 17, the therapeutic agent being provided for the therapy or prevention of Segawa syndrome; spontaneous dyskinesia or dystonia associated with Parkinson's disease or tardive or L-DOPA induced dyskinesia or dystonia.

19. The use of a compound of the general formula (III): 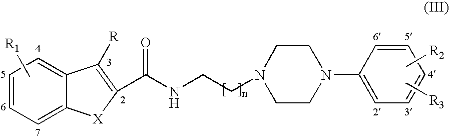
wherein:
n=1-4 and X=S, O or NH, when R=hydrogen, alkyl or halogen and R₁ is substituted by the radicals hydrogen, alkyl, halogen, trifluoromethyl and each of R₂ and R₃ are substituted individually or jointly by the radicals hydrogen, hydroxy, alkyloxy, alkyl, alkenyl, alkinyl, aryl, halogen, trifluoromethyl, acyl, alkoxycarbonyl or cyano,
for preparing a pharmaceutical agent for the therapy or prevention of cocaine, alcohol, opiate and nicotine addiction; neurodegenerative disorders, especially Parkinson's disease; or sexual dysfunction.

20. The use of a compound according to claim 19 for preparing a therapeutic agent for the therapy or prevention of depression or schizophrenia.

21. The use of a compound according to claim 19 for preparing a therapeutic agent for the therapy or prevention of hyperprolactinaemia; hyperprolactinoma; glaucoma; cognitive disorders; restless leg syndrome; hyperactivity syndrome (ADHS); locomotion disorders associated with Parkinson's disease; L-DOPA-induced disorders, Segawa syndrome; tardive locomotion disorders as well as for medication-assisted ablactation after pregnancies.

22. The use according to claim 21, the therapeutic agent being used for the therapy or prevention of Segawa syndrome, spontaneous dyskinesia or dystonia associated with Parkinson's disease or tardive or L-DOPA induced dyskinesia or dystonia.

23. The use according to claim 19 wherein
R is selected from hydrogen, C₁-C₆ alkyl, fluorine, chlorine and bromine,
R₁ is selected from hydrogen, C₁-C₆ alkoxy, C₁-C₆ alkyl, fluorine, chlorine, bromine and trifluoromethyl, and
each of R₂ and R₃ is independently selected from hydrogen, C₁-C₆ alkoxy, C₁-C₆ alkyl, fluorine, chlorine, bromine and trifluoromethyl
wherein the groups C₁-C₆ alkyl may optionally also be substituted.

24. The use according to claim 19, wherein
the substituent R₁ is in position 5 or 6 of the heterocycle, and
the substituents R₂ and R₃ are in the positions 2 or 3, respectively, or in the positions 2 or 4, respectively, of the phenyl ring; the respective other substituent being in position 2 of the phenyl ring in the event that one of the two substituents R₂ and R₃ is a hydrogen atom.

25. The use according to claim 19 wherein the compound is N-4-(4-(2-methoxyphenyl)piperazine-1-yl)butyl-2-indolylcarbamide.

26. A method for preparing a compound of the general formulae (I), (III), or (IV) as defined above comprising reacting a compound of the general formula (A) in activated form, especially in the form of the carboxylic acid halide 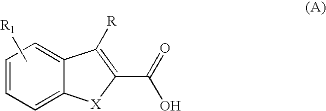
with a compound of the general formula (B): 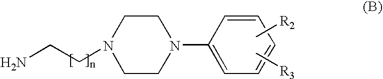
wherein n, R, R₁, R₂ and R₃ are as defined for the general formulae (I), (III) and (IV).

27. A method for preparing a compound of the general formula (II) as defined above comprising reacting ferrocene-2-carboxylic acid in activated form with a compound of the general formula (B′) 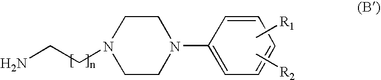
wherein n, R₁ and R₂ are as defined in formula (II).
